(19) United States
(12) Patent Application Publication
(10) Pub. No.: US 2012/0064041 A1
(43) Pub. Date: Mar. 15, 2012

(54) Efficient Generation of Neurally-Induced Mesenchymal Stem Cells and Applications Thereof

(76) Inventor: Arshak Alexanian, Milwaukee, WI (US)

(21) Appl. No.: 13/225,441

(22) Filed: Sep. 3, 2011

Related U.S. Application Data
(60) Provisional application No. 61/380,114, filed on Sep. 3, 2010.

(51) Int. Cl. A61K 35/12; C12N 5/0789; C12N 5/0797; A61K 35/34; A61P 25/00; C12N 5/0775; A61K 35/14

(52) U.S. Cl. 424/93.7; 435/377; 435/372; 435/371; 435/366

(57) ABSTRACT

The present invention provides methods and compositions for reprogramming mammalian mesenchymal stem cells, as well as to methods for using such cells, for example, to prevent or treat various injuries, diseases, and disorders in human and non-human animals.

### RELATED APPLICATION

This application claims the benefit of and priority to U.S. provisional patent application Ser. No. 61/380,114, filed 3 Sep. 2010, which is commonly owned with the instant application and is herein incorporated by reference in its entirety for any and all purposes.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art or even particularly relevant to the presently claimed invention.

2. Background

Stem cell-based therapy to repair and replace lost neural cells is a highly promising treatment for central nervous system (CNS) diseases. Bone marrow (BM)-derived mesenchymal stem cells (MSCs) have great potential as therapeutic agents against neurological maladies since they have the ability to differentiate into neural phenotypes and can be readily isolated and expanded for auto-transplantation with no risk of rejection. Within the past several years, MSCs have been considered an important source of stem cells for cell therapy and regeneration.

One advantage of using MSCs for cell therapy is their ability to be isolated and expanded from different adult and postnatal tissues, such as BM [Prockop, D J, Science 276(5309), 71-74 (1997)], peripheral blood [Kuznetsov, et al., J Cell Biol 153(5), 1133-1140 (2001)], muscle [Lee, et al., J Cell Biol 150(5), 1085-1100 (2000)], vasculature [Brighton, et al., Clin Orthop (275), 287-299 (1992)], skin [Mizuno and Glowacki, Exp Cell Res 227(1), 89-97 (1996)], adipose tissue [Zuk, et al., Tissue Eng 7(2), 211-228 (2001)], and umbilical cord [Lee, et al., Blood 103(5), 1669-1675 (2004)]. Because of the ease of isolation and expansion, MSCs could be an ideal source for autologous and allogeneic transplantation.

Another advantage of MSCs is the ability to differentiate into multiple cell types of mesodermal, endodermal, and epidermal origin such as bone [Pereira, et al., Proc Natl Acad Sci USA 92(11), 4857-4861 (1995)], cartilage [Pereira, et al., Proc Natl Acad Sci USA 95(3), 1142-1147 (1998)], fat [Umezawa, et al., Mol Cell Biol 11(2), 920-927 (1991)], muscle [Ferrari, et al., Science 279(5356), 1528-1530 (1998)], cardiomyocytes [Makino, et al., J Clin Invest 103(5), 697-705 (1999)], and neurons [Kohyama, et al., Differentiation 68(4-5), 235-244 (2001)]. Demonstration of neural differentiation potential of MSCs in several in vitro and in vivo studies suggests the potential usefulness of MSCs in the treatment of various CNS disorders. This potential has led to extensive studies to further explore the neural plasticity of these cells [Kohyama, et al., Differentiation 68(4-5), 235-244 (2001); Kopen, et al., Proc Natl Acad Sci USA 96(19), 10711-10716 (1999); Munoz-Elias, et al., J Neurosci 24(19), 4585-4595 (2004)].

During the last several years, numerous in vitro neural induction protocols to produce neural cells from MSCs have been reported. In most induction experiments, MSCs were simply exposed to neurotrophic factors or factors favoring neural cell growth and differentiation [Kim, et al., Neuroreport 16(12), 1357-1361 (2005); Sanchez-Ramos, et al., Exp Neurol 164(2), 247-256 (2000); Kim, et al., Neuroreport 13(9), 1185-1188 (2002); Joannides, et al., J Hematother Stem Cell Res 12(6), 681-688 (2003); Padovan C S, et al., Cell Transplant 12(8), 839-848 (2003); Kondo, et al., Proc Natl Acad Sci USA 102(13), 4789-4794 (2005); Chen, et al., J Neurosci Res 80(5), 611-619 (2005); Long, et al., Stem Cells Dev 14(1), 65-69 (2005)]. Other studies have used different culture media, supplemented with individual or various combinations of chemical and pharmacological agents, such as DMSO, b-mercaptoethanol, 5-bromo-2-deoxyuridine (BrdU), butylated hydroxyanisole, forskolin, and dibutyryl cyclic AMP [Kim, et al. (2005), above; Lu, et al., J Neurosci Res 77(2), 174-191 (2004); Yang, et al., Zhonghua Yi Xue Za Zhi 85(16), 1125-1128 (2005); Munoz-Elias, et al., Stem Cells 21(4), 437-448 (2003); Deng, et al., Biochem Biophys Res Commun 282(1), 148-152 (2001); Hermann, et al., J Neurosci Res 83(8), 1502-1514 (2006); Episkopou, V, Trends Neurosci 28(5), 219-221 (2005); Ankeny, et al., Exp Neurol 190(1), 17-31 (2004); Jori, et al., J Cell Biochem 94(4), 645-655 (2005)]. Other methods to induce MSCs into cells with neural characteristics include: transfection of MSCs with Noggin and Notch transcription factors [Kohyama, et al., (2001), above; Dezawa, et al., J Clin Invest 113(12), 1701-1710 (2004)]; manipulation with surface proteins of culture substrate [Qian and Saltzman, Biomaterials 25(7-8), 1331-1337 (2004)]; co-culturing MSCs with neural stem cells (NSCs) or neural cells [Jiang, et al., Proc Natl Acad Sci USA 100 Suppl 1, 11854-11860 (2003); Wislet-Gendebien, et al., Stem Cells 23(3), 392-402 (2005); Alexanian, A R, Exp Cell Res 310(2), 383-391 (2005); Krampera, et al. Bone 40(2), 382-390 (2007)]; and growing MSCs as spheres in cultures [Shiota, et al., Exp Cell Res 313(5), 1008-1023 (2007)]. In several other studies, MSCs were converted into more specific multipotent cells and then induced into neural cell lineages, by exposing them to appropriate neural differentiation conditions [Kohyama, et al., (2001), above; Qu, et al., Restor Neurol Neurosci 22(6), 459-468 (2004)].

Despite such work, debate continues about the nature of the reported differentiation responses. For example, it has been suggested that cell fusion could account for transdifferentiation [Terada, et al., Nature 416(6880), 542-545 (2002)]. However, spontaneous cell fusion occurs only very rarely and thus likely cannot account for massive transdifferentiation demonstrated in numerous recent studies. In addition, MSCs can be induced into neural-like cells with several neural inducing factors, without being grown in co-cultures with NSCs. Similarly, it has been suggested that some of the reported differentiation responses were artifacts created by in vitro chemical stress [Lu, et al., (2004), above; Neuhuber, et al., J Neurosci Res 77(2), 192-204 (2004)]. Numerous other studies, however, suggest that with appropriate neural induction protocols, MSCs could produce mature neuron-like cells that exhibit multiple neuronal properties and traits, such as action potential, synaptic transmission, secretion of neurotrophic factors and dopamine, and demonstration of spontaneous post-synaptic current [Jiang, et al., (2003) above; Wislet-Gendebien, et al., (2005), above; Mareschi, et al., Cytotherapy 11(5), 534-547 (2009); Bonilla, et al. Neuroscience 133(1), 85-95 (2005); Kim, et al, Stem Cells 26(9), 2217-2228 (2008); Trzaska, et al., J Neurochem 110(3), 1058-1069 (2009); Alexanian, et al., Stem Cells Dev 17(6), 1123-1130 (2008); Hermann, et al., J Cell Sci 117(Pt 19), 4411-4422 (2004).

Notwithstanding the significant effort made to date, a significant need remains for a more efficient procedure for the generation of neural stem cells from MSCs, and the development of new techniques to differentiate them into specific glial or neuronal phenotypes.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “combination therapy” refers to a therapeutic regimen that involves the provision of at least two distinct therapies to achieve an indicated therapeutic effect. For example, a combination therapy may involve the administration of two or more chemically distinct active ingredients, for example, an isolated cell population according to the invention and one or more biological and/or chemical agents. Combination therapy may, alternatively, involve administration of an isolated cell population according to the invention with the delivery of another treatment, such as radiation therapy and/or surgery. In the context of combination therapy using two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when an isolated cell population according to the invention is administered, alone or in conjunction with one or more other drugs and/or radiation and/or surgery, the cells and drug(s) may be delivered before or after surgery or radiation treatment.

“Monotherapy” refers to a treatment regimen based on the delivery of one therapeutically effective composition, whether administered as a single dose or several doses over time.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

A “plurality” means more than one.

The terms “separated,” “purified,” “isolated,” and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, unreacted chemicals, proteins, carbohydrates, lipids, cells, cell fragments or organelles, and unbound molecules.

The term “species” is used herein in various contexts, e.g., a particular species of biological or chemical agent, cell type, etc. In each context, the term refers to a population of cells or molecules, chemically indistinguishable from each other, of the sort referred in the particular context.

A “subject” or “patient” refers to an animal in which treatment can be effected in accordance with the invention. The animal may have, be at risk for, or be believed to have or be at risk for a disease or condition that can be treated by compositions and/or methods of the present invention. Animals that can be treated in accordance with the invention include vertebrates, with mammals such as bovine, canine, equine, feline, ovine, porcine, and primate (including humans and non-human primates) animals being particularly preferred examples.

A “therapeutically effective amount” (or “effective amount”) refers to an amount of an active ingredient, e.g., an isolated cell population according to the invention, sufficient to effect treatment when administered to a subject or patient. Accordingly, what constitutes a therapeutically effective amount of a cellular composition according to the invention may be readily determined by one of ordinary skill in the art. Of course, the therapeutically effective amount will vary depending upon the disease or condition and patient being treated, the weight and age of the subject, the severity of the disease condition, the particular composition to be administered or otherwise delivered, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (ie., a therapeutic regimen that employs only one chemical entity as the active ingredient).

The term “treatment” or “treating” of a disease or disorder includes preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

The term “therapeutic regimen” means any treatment of a disease or disorder using one more biologic or chemical (i.e., small molecule) drugs, radiation therapy, surgery, gene therapy, DNA vaccines and therapy, antisense-based therapies including RNAi or siRNA therapy, anti-angiogenic therapy, deliver of an isolated cell population according to the invention, immunotherapy, bone marrow transplants, aptamers and other biologics such as antibodies and antibody variants, receptor decoys, and other protein-based therapeutics.

### SUMMARY OF THE INVENTION

Stem cells derived from embryonic, postnatal, and adult tissues have emerged as potential sources of cells for ceil-based therapies that could revolutionize the treatment of a wide range of diseases, including neurological disorders. Recent advances in generating multi- or pluripotent stem cells from adult somatic cells, or by transdifferentiating one type of adult stem cell to another, make it possible to generate desirable tissue-specific stem cells from a patient's own somatic cells. This nascent technology, however, requires safer, easier, and more efficient procedures for the generation of desired cell phenotypes via cell reprogramming. Additionally, sources of cells from adult tissues that can be obtained readily and reprogrammed easily and efficiently is another important issue for the development of such technologies. Therefore, the identification of an easily accessible source of cells with relatively higher plasticity and the development of technologies to manipulate them into desired cell types will enable cell-based clinical therapies for the treatment of a tremendous number of diseases and conditions in humans and non-human animals.

Mesenchymal stem cells (MSCs) are an important source of stem cells for cell therapy and regeneration, particularly since they can be isolated and expanded from different adult and postnatal tissues, such as bone marrow, peripheral blood, muscle, vasculature, skin, adipose tissue, and umbilical cord as well as differentiate into multiple cell types of mesodermal, endodermal, and epidermal origin, for example, as bone, cartilage, fat, muscle, cardiomyocytes, and neurons.

The present invention is directed to methods for generating reprogrammed mammalian mesenchymal stem cells, particularly human MSCs. In preferred embodiments, such cells are then directed to neural lineages so as to be useful in treating spinal cord or other central nervous system injuries or diseases.

Thus, in one aspect, the invention concerns methods for reprogramming mammalian mesenchymal stem cells. Generally, these methods comprise culturing cells that include mammalian mesenchymal stem cells under conditions that include an effective amount a first epigenetic modifying agent, a second epigenetic modifying agent, and a cAMP elevating agent for a period sufficient to allow reprogramming of at least a portion of the mammalian mesenchymal stem cells. In preferred embodiments, the mammalian mesenchymal stem cells are human mesenchymal stem cells, which can be isolated from any suitable source or tissue, including bone marrow, peripheral blood, muscle, vasculature, skin, adipose tissue, or umbilical cord.

Typically, one of the epigenetic modifying agent species is an inhibitor of DNA methylation, with 5-aza-2′-deoxycytidine (5azadc) and RG-108 being particularly preferred DNA methylation inhibitors. RG-108 is preferably used in a ranging from about 1 μM to about 10 μM. The other epigenetic modifying agent species is an inhibitor of histone deacetylation, preferably Trichostatin A (TSA). Particularly preferred TSA concentrations range from about 50 nM to about 500 nM. Additional or different epigenetic modifying agents may also be employed.

With regard to the cAMP elevating agent, it can be any agent or compound that can result in intracellular elevation of cAMP levels. Particularly preferred are hydrolysis-resistant forms of cAMP, for example, BrcAMP, adenylate cyclase activators (e.g., Forskolin), and inhibitors of cAMP phosphodiesterase such as IBMX or Rolipram. Particularly preferred concentrations for 8-BrcAMP range from about 100 μM to about 500 μM, and for Rolipram, from about 0.1 μM to about 10 μM.

The cells are cultured for a sufficient period under suitable culturing conditions to allow reprogramming to occur, usually from about 5-20 days. In certain preferred embodiments, the cells are cultured under conditions that not only favor reprogramming, but also induction toward particular cell lineages or types. For example, in some embodiments, induction of neural or neural-like cells is desired, particularly if the cells are to be used for cell therapy to treat a central nervous system disease or disorder or a =spinal cord injury. In such instances, growth factors, drugs, and/or other compounds that promote neural differentiation can be included during the cell culture process. A particularly preferred growth factor in the context of the invention for inducing neural differentiation is bFGF. When neural induction is desired, the methods can include one or more neural induction factors (e.g., bFGF) during cell culture. When bFGF is used, particularly preferred culture concentrations range from about 5 ng/mL to about 50 ng/mL.

Related aspects of the invention concern cell populations produced according to the methods of the invention, including reprogrammed mammalian mesenchymal stem cells neural stem cells. Yet other related aspects are directed to methods of using such cell populations, for example, for various cell therapies, drug screening, or as research tools. In the treatment context, particularly preferred applications for neural stem cells produced in accordance with the instant methods include treating spinal cord injuries or central nervous system diseases or conditions. Such methods involve administering to a subject having or suspected of having a spinal cord injury or central nervous system disease or condition a cell population that includes neural stem cells produced as described herein. Preferably, such cell populations comprise at least about 10%, preferably more than at least about 25%, about 50%, 80%, 90% or more neural stem cells. The therapeutic cell population can be delivered to the subject by any suitable route. Preferred routes include intravenous, intrathecal, or direct administration into the injured spinal cord or central nervous system tissue.

These and other aspects and embodiments of the invention are discussed in greater detail in the sections that follow.

### BRIEF DESCRIPTION OF THE FIGURES

This application contains at least one figure executed in color. Copies of this application with color drawing(s) will be provided upon request and payment of the necessary fee. A brief summary of each of the figures is provided below.

FIGS. 1-3 show results from experiments described in Example 1, below. FIG. 1 has three panels (a)-(c) (left to right). Human MSCs from P1 (a) grown in adiopogenic or osteoblastic induction media differentiated into fat (b) and bone (c). Adipogenesis was evaluated by Oil Red O staining and osteogenesis by Alizarin Red staining.

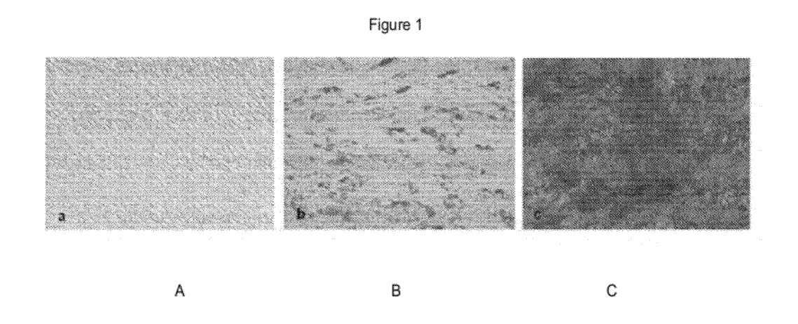

FIG. 2 shows expression of neural markers nestin, Sox2, A2B5, NCAM, B3T, GFAP, MAP-2, and NeuN in hMSCs (a-d) and NI-hMSC grown 24 h, 1, 2, 3 weeks in neural induction medium (e-t). NI-hMSCs grown an additional week in neuronal induction medium were generated cells with long axon- and dendrite-like extensions (v-w). Bars represent a distance of 40 um.

FIG. 3 shows Western blot results for the expression of neural markers B3T, Sox-2, GFAP, MAP2, and NeuN in untreated hMSCs and in NI-hMSCs after 3 weeks of treatment (A). Real time RT-PCR for neural- and pluripotency-associated genes in MSC before and after 24, 1, 2 and 3-weeks of neural induction (B,D). Electrophoresis of PCR products on 2% agarose gel were visualized by ethidium bromide staining (C).

FIGS. 4-8 show results from the experiments described in Example 2, below. FIG. 2 shows the expression of several neural markers in NI-hMSC grown for 2 weeks in neural induction medium.

FIG. 5 shows a plot of locomotor recovery (BBB) scores for a post spinal cord injury (DPI-days-post-injury) behavioral analysis. The asterisks (*) and (**) indicate significant differences between the NI-hMSC-transplanted group compared to the PBS and PBS+hMSC control groups, respectively. Asterisk (***) indicates a significant difference between the hMSC-transplanted group compared to the PBS control.

FIG. 6 shows two graphs representing hindlimb (A) and forelimb (B) behavioural responses recorded as paw withdrawal latencies to thermal stimulation.

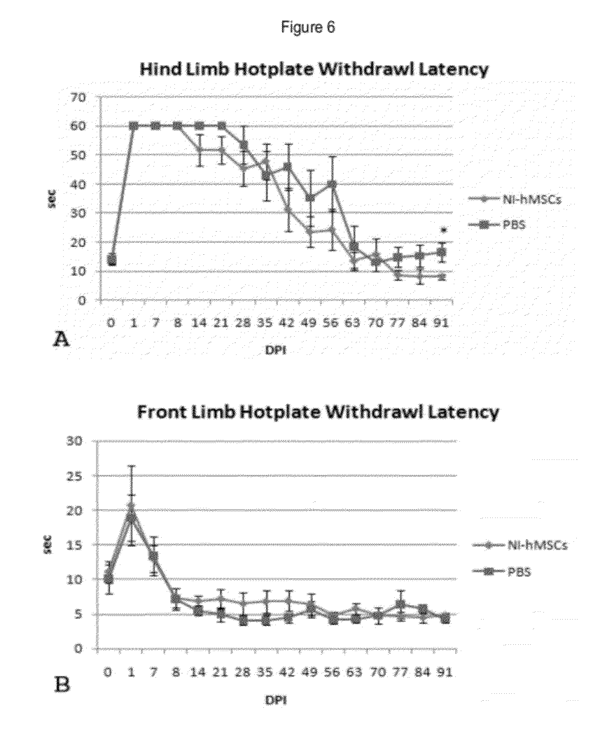

FIG. 7 shows nine color images. Transplanted NI-hMSCs survived 2 weeks after transplantation and expressed neural markers such as B3T (images a-c; images b and c are the higher magnifications of the marked area in the image a) and GFAP (images d-f). By 12 weeks the number of surviving cells declined to 15-20% of that at week 2 and only 10% of survived cells were positive to B3T (images g, h, i). The images h and i are higher magnifications of g. HM stands for human anti-mitochondrial antibody

FIG. 8 has three panels showing analyses of white matter sparing and lesion cavity volumes in NI-hMSCs, hMSCs, and PBS treated groups. (A) shows representative spinal cord cross-sections extending 500 um rostral and caudal from the lesion epicenter. (B) is a graph representing the percentages of spared white matter through the entire T8 spinal cord segment. (C) is a graph representing comparison of the volumes of lesion cavities.

FIG. 9 shows the morphological and immunocytochemical characterization of unmodified and NI-fMSCs (see Example 3, below). Expression of neural markers B3T, NCAM, A2B5, MAP2, NeuN, NF, Nurr1, TH and ChAT in unmodified fMSCs (a-f) and in NI-fMSCs grown for 72 h in neural induction medium (g-r).

FIG. 10 is a graph showing the results of RT-PCR analysis of pluripotent gene expression in unmodified feline MSCs and in NI-fMSCs after 24 h, 48 h, and 72 h of treatment (see Example 3, below). Expression levels of Sox2, Klf4, Nanog, and Oct4 were gradually increased over time and most of them were significantly different from the control after 24 h of the treatment. The expression level of cMyc was significantly increased after 24 h of the treatment but decline during the next two days.

FIG. 11 shows data from RT-PCR analysis of immature and mature neural gene expression in unmodified fMSCs and in NI-fMSCs after 24 h, 48 h, and 72 h of treatment (see Example 3, below).

FIG. 12 shows Western blot analysis (see Example 3, below) of pluripotent gene expression in unmodified fMSCs and in NI-fMSCs after 24 h, 48 h, and 72 h of treatment. In FIG. 7, expression of genes normalized to b-actin (image on the left), while the graph in the right panel shows the expression levels of Sox2, Nanog, and cMyc in treated cultures were significantly different from untreated fMSCs after 48 or 72 h of the treatment.

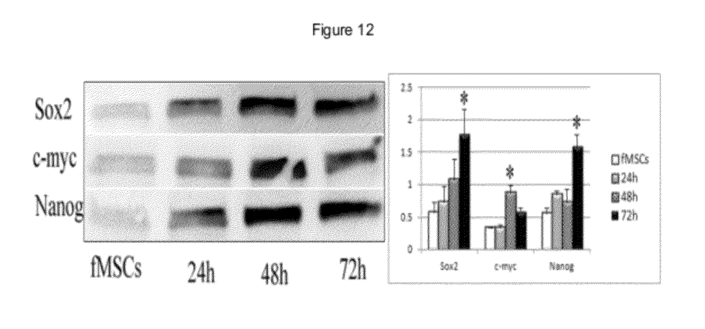

FIG. 13 shows Western blot analysis (see Example 3, below) of neural gene expression in unmodified fMSCs and in NI-fMSCs after 24 h, 48 h, and 72 h of treatment. Expression of genes normalized to b-actin (image of the left). The expression levels of neural genes in NI-hMSCs were significantly higher from untreated hMSCs after 48 h and/or 74 h of the treatment.

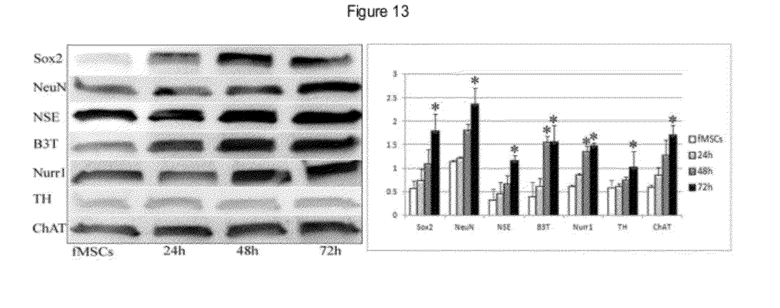

### DETAILED DESCRIPTION OF THE INVENTION

### In General

During the last two decades, stem cells have become recognized as promising tools for various biomedical applications, including disease modeling, drug development, and cell replacement therapies, as stem cells can undergo self-renewing cell division to give rise to phenotypically and genotypically identical daughters for an indefinite time and ultimately differentiate into at least one, and often many, final cell type(s). As such, stem cells can repopulate tissues upon transplantation. The quintessential stem cell is the embryonal stem (ES) cell, as it has unlimited self-renewal and pluripotent differentiation potential. Stem cells have also been identified in several organ tissues. These include hematopoietic stem cells, neural stem cells, and mesenchymal stem cell (MSCs). However, identification of the reliable sources of stem cells that can be easily harvested, expanded on a large enough scale, and carry no risk of immune rejection still remains one of the important issues for regenerative medicine.

As is known, during normal development of multicellular organisms, different cells and tissues exhibit different patterns of gene expression. These distinct cell- and tissue-specific patterns are primarily regulated by epigenetic modifications, such as DNA methylation, histone modifications, and the association of various chromatin-binding proteins to chromosomal DNA. Thus, each cell type has a characteristic epigenetic signature that becomes “fixed” as cells differentiate or lose the capacity to divide. Some cells, however, undergo epigenetic “reprogramming” during normal development or in certain diseases. Such “reprogramming” involves removing epigenetic features in the nucleus, followed by establishment of a different set epigenetic modifications characteristic of a different cell type or lineage. For example, at fertilization, gametic epigenetic features are removed and replaced with features required for totipotency and embryonic development. Reprogramming also occurs in primordial germ cells, where parental imprints are erased to restore totipotency. Cancer cells and cells that transdifferentiate also are thought to undergo reprogramming.

The instant invention is based on the discovery of methods for efficiently generating stem cells to particular cell lineages from more primitive reprogrammed pluripotent stem cells. In the context of the invention, reprogramming results in transient changes to DNA and chromatin structure, not in mutations. Thus, reprogramming results in removing existing epigenetic features, followed by establishing epigenetic features characteristic of different cell types by transiently altering epigenetic features during the cell reprogramming phase and then providing one or more compounds to preferentially drive differentiation toward the desired cell lineage(s). In particularly preferred embodiments, this discovery has been extended to achieve neural induction of mammalian mesenchymal stem cells, particularly human bone marrow-derived MSCs (hMSCs), which leads to generation of neural stem cells. Neural induction of BM-hMSCs can be achieved, for example, by exposing hMSCs simultaneously to inhibitors of DNA methylation and histone deacetylation, along with agents that can elevate cAMP levels. Further extending the invention is the discovery of neural stem cells generated according to the instant neural induction methods can survive, differentiate, and significantly improve locomotor recovery of injured spinal cord in vivo.

Generation of a Population of Neural Cells from hMSCs

In one aspect, the invention concerns methods of generating neurally-induced mammalian MSCs, particularly neurally-induced human MSCs (NI-hMSCs), from human or other mammalian MSCs. This is done by exposing the MSCs isolated from any suitable source to a combination of at least two epigenetic modifiers and at least one agent that can elevate intracellular cyclic adenosine monophosphate (cAMP) levels. In a particularly preferred embodiment, the cells are also exposed to bFGF. Such treatments lead to reversion of mammalian MSCs to a less differentiated, preferably embryonic-like progenitor cell state followed by re-differentiation into or toward a desired tissue, cell, or lineage, for example, neural or neural-like cells. In particularly preferred embodiments the cells are NI-hMSCs, also referred to herein as human “neural stem cells”. The efficiency of the invention is borne out in that, in the context of NI-hMSCs, after 2-3 weeks of neural induction approximately 95% of cells express several neural markers and 20-30% percent of these neurally modified cells produce long axon- and dendrite-like extensions.

Molecules useful in “reprogramming” cells can be naturally occurring or synthetic, and include proteins (e.g., growth factors, hormones, etc.), peptides, nucleic acids (e.g., antisense nucleic acids, small interfering RNA molecules, etc.), and small molecule chemical compounds. It will also be appreciated that other molecules or agents, or combinations of molecules, can be used in place of those specified herein, and it is understood that such molecules or agents will be deemed to be equivalents for those substituted thereby. Identifying substitutes or equivalents can be performed by any suitable assay, and once identified, comparisons can be with the various aspects and embodiments of the invention.

In the context of the invention, “hMSCs”, refer multipotent stem cells obtained from any suitable source. For example, hMSCs can be obtained via a colony-forming unit-fibroblasts (CFU-f) approach. In a CFU-f approach, raw, unpurified bone marrow is directly plated onto a cell culture surface, such, as for example, plastic plates, culture flasks, or plastic beads. Alternatively, ficoll-purification can be used to obtain ficoll-purified bone marrow monocytes that can then be directly plated onto a cell culture surface. Human MSCs are characterized by their ability to adhere to a cell culture surface within 24 to 48 hours. By contrast, neither red blood cells nor hematopoetic progenitors adhere to the cell culture surface within 24 to 48 hours.

Also, although hMSCs are utilized in a variety of biomedical applications, there is a lack of consensus on the criteria for distinguishing MSCs. Accordingly, MSC identification relies on a combination of positively and negatively expressed markers that facilitates their characterization among other cellular subsets. First, as noted above, MSC must be plastic-adherent when maintained in standard culture conditions. Second, MSC must express CD105, CD73, and CD90, and lack CD45, CD34, CD14 (or CD11b), CD79a (or CD19), and HLA-DR surface molecules. Lastly, under standard conditions, MSC must be capable of differentiating to osteoblasts, adipocytes, and chondroblasts in vitro.

An “embryonic-like progenitor cell” refers to intermediate-type cells that start out as hMSCs, then because of exposure to a combination of epigenetic modifiers and agents that elevate cAMP levels, the cells de-differentiate (revert back or are “re-programmed”) to a pluripotent cell type, characterized by the expression of at least three pluripotency-associated genes selected from the group consisting of Oct-4, Nanog, Klf4, c-Myc, and Sox-2. Subsequently, these intermediate, embryonic-like progenitor cells re-differentiate into a highly pure population of cells, wherein at least 70% and preferably 80% of the cells are neural stem cells. In some embodiments, biological factors such as bFGF, alone or in combination with other factors that can induce neural differentiation, are also included in the culture medium to promote neural differentiation. The resultant population of neural stem cells can be directly transplanted, for example, into a spinal cord or other central nervous system tissue to effect treatment.

In the context of the invention, “neural stem cells” refers to a population of cells encompassing both immature and mature neural stem cells. Specifically, immature neural stem cells express at least two neural cell progenitor markers, such as, Sox2, nestin, A2B5, and NCAM. These immature neural stem cells are able to further differentiate into mature neural stem cells that are positive for the expression of at least two mature neural markers, such as B3T, GFAP, Olig1, O1, O4, NeuN, NSE, NP200, and MAP2. These differentiated neural stem cells release the neurotrophic factors GDNF and BDNF. The neural stem cells are also capable of surviving and differentiating to promote functional recovery of injured spinal cord or other CNS tissue when transplanted into an injured spinal cord, brain, etc. Here, “functional recovery”, means recovery in locomotor and sensory functions, cognitive function, etc., as the case may be in the context of the particular treatment.

In this invention, an “epigenetic modifier” or “epigenetic modifying agent” is an inhibitor of DNA methylation or histone deacetylation. DNA methylation inhibitors include but are not limited to 5-aza-2′-deoxycytidine (5azadc) and RG-108 (N-Phthalyl-L-tryptophan). Histone deacetylation inhibitors include but are not limited to Trichostatin A (TSA; 7-[4-(dimethylamino)phenyl]-N-hydroxy-4,6-dimethyl-7-oxohepta-2,4-dienamide). A cAMP elevating agent is any agent capable of elevating intracellular AMP levels. Agents that can elevate cAMP levels include but are not limited to BrcAMP (8-Bromoadenosine-3′,5′-cyclic monophosphate; a hydrolysis-resistant form of cAMP), Forskolin (an adenylate cyclase activator), and IBMX (3-isobutyl-1-methylxanthine) and Rolipram ((RS)-4-(3-cyclopentyloxy-4-methoxy-phenyl)pyrrolidin-2-one) (each an inhibitor of cAMP phosphodiesterase). Basic fibroblast growth factor, “bFGF”, is a growth factor that involved in the regulation of numerous cellular processes. bFGF is often used as a component of human embryonic stem cell culture medium. It is also preferably used to help cells to remain in an undifferentiated state and induce gremlin expression, which in turn is known to inhibit the induction of differentiation by bone morphogenetic proteins. It is also a factor in neural differentiation.

In some preferred embodiments of this aspect of this invention, the hMSCs are treated with TSA, RG-108, 8-BrcAMPn and/or Rolipram. Preferably, the concentration of TSA ranges from about 1-1000 nM, more preferably from about 50-500 nM, with about 200 nM being particularly preferred. Preferably, the concentration of RG-108 ranges from about 0.01-100 μM, even more preferably, from about 1-10 μM, with about 3 μM being particularly preferred. Preferred concentrations of 8-BrcAMP range from about 0.5-1000 μM, more preferably from about 50-500 μM, with about 300 μM being especially preferred. Preferred concentrations of Rolipram ranges from about 0.01-100 μM, preferably 0.1-10 μM, and even more preferably, 1 μM. When bFGF is included, bFGF concentrations range from about 0.05-250 ng/mL, preferably, from about 5-50 ng/mL, and even more preferably, about 20 ng/mL.

In preferred embodiments, hMSCs are treated with a combination of epigenetic modifiers and at least one agent that can elevate cAMP levels, and preferably bFGF, for at least about 5 to about 20 days.

In some embodiments, the reprogrammed mammalian mesenchymal stem cells or neural stem cells produced in accordance with the invention may or may not be genetically engineered, depending on the particular therapy being delivered to the patient. In this context, “genetically engineered” refers to cells that have been intentionally engineered to contain one or more heritable genetic modifications in one or more chromosomes, as those in the art will appreciate.

A related aspect concerns populations of neural stem cells generated by the methods of the present invention. Preferably, the neural stem cells release the neurotrophic factors GDNF and BDNF and express at least four neural markers selected from the group consisting of Sox2, nestin, A2B5, NCAM, B3T, GFAP, NeuN, NSE, NF200, and MAP2. Preferably, at least about 50%, more preferably at least about 80%, of the cells in the population are neural stem cells. Particularly preferred are populations where at least about 90-95% of the cell population is neural stem cells.

### Transplantation Therapy for Spinal Cord Injury

Injury to the human spinal cord produces a complex pathology that currently lacks a clinical treatment regimen capable of promoting significant anatomical repair and functional restoration. Thus, the development of new strategies to treat such injuries is a major clinical challenge. The ability to transplant stem/progenitor cells offers a novel and exciting possibility for repairing injured nervous tissue of spinal cord through replacement of damaged cells, neuroprotection, or the creation of an environment conducive to regeneration by endogenous cells.

Another aspect of this invention relates to methods of treating spinal cord injury or other central nervous system conditions by transplanting an effective amount of neural stem cells generated via the methods described above into a human or non-human animal subject. Here, “treating” refers to transplanting an amount of neural stem cells (i.e., a “population of neural stem cells”) that will be effective to promote recovery in locomotor and sensory functions of the injured spinal cord or other damaged or injured tissue of the central nervous system.

This invention envisions treatments that use cell populations of the invention that are allogeneic, autologous, or syngenic. An “allogeneic” cell therapy refers to a procedure where a subject having a spinal cord or other CNS injury or disease amenable to a cell therapy treatment (including providing support to CNS tissue) receives cells from a genetically similar but not identical donor. Genetic similarity is often based on HLA-typing. An “autologous” cell therapy refers to a procedure where the subject donates her/his own cells. A “syngeneic” cell therapy refers to a procedure where a subject receives cells from a genetically identical donor, i.e., from an identical twin. Autologous and syngeneic therapies avoid problems associated with immune rejection of transplanted cells or tissues. As will be appreciated

In some preferred embodiments, MSCs are isolated from the human or animal subject suffering from a spinal cord injury or a central nervous system condition. The hMSCs are then expanded. For stem cells, or even fully differentiated cell populations to be truly beneficial or useful, either therapeutically, as drug screening tools, or for core research purposes, expansion of cells is prerequisite. An appropriate culture environment is critical to stem cell expansion efforts. This environment is typically maintained by a combination of media, supplements, and reagents. The expanded hMSCs are then exposed to a combination of at least two epigenetic modifiers and at least one agent that elevates cAMP levels. Preferably, the cells are also exposed to bFGF. As a result of such transient chromatin modifying, reprogramming procedures, neural stem cells are produced. Populations of these neural stem cells are then transplanted into the spinal cord having an injury. One could transplant the neural stem cells intravenously, intrathecally, or directly into the injured spinal cord. It is envisioned that the neural stem cells produced according to the invention can also be used to treat conditions of the central nervous system by direct transplantation into central nervous system tissue. After implantation, additional rounds of cell therapy can be provided, if desired. Cell therapies may also involve or be part of combination therapies wherein one or more other neurotropic factors (e.g., BDNF, GDNF) are also administered to the subject as part of the desired treatment.

### EXAMPLES

The following preparations and examples are given to enable those skilled in the art to more clearly understand and to practice the present invention. They should not be considered as limiting the scope of the invention, but merely as being illustrative and representative thereof.

### Example 1

### Efficient Methods for Generating Neural-Like Cells from Adult Human Bone Marrow-Derived Mesenchymal Stem Cells

### 1. Introduction

Stem cell-based therapies to repair and replace lost neural cells represent highly promising treatments for central nervous system (CNS) diseases. Mesenchymal stem cells (MSCs), particularly those derived from bone marrow (BM), can serve as therapeutic agents to treat a wide range of neurological maladies since they have the ability to differentiate into a range of neural phenotypes and can be readily isolated and expanded for allogeneic, autologous, or syngeneic transplantation. This example describes an approach to efficiently generate neural-like (NI) cells from human BM-derived MSCs by exposing BM-hMSCs to epigenetic modifiers in a neural environment in order to reactivate pluripotency-associated genes in the BM-hMSCs before or while also exposing them to neural inducing factors. Briefly, neural induction was achieved here by simultaneously exposing BM-hMSCs to inhibitors of DNA methylation and histone deacetylation and one or more pharmacological agents that increase cAMP levels. The expression of pluripotency and neural markers was confirmed by immunocytochemistry, Western blot, and RT-PCR analyses. ELISA studies showed that these NI-hMSCs cells released the neurotrophic factors GDNF and BDNF. The results demonstrate hMSCs that are modified using such methods can be useful sources of cells for CNS repair and regeneration.

### 2. Experimental Procedures

A. Expansion of hMSCs. Human bone marrow MSCs (frozen at passage 1) were obtained from Tulane University Center of Gene Therapy (grant from NCRR of the NIH, Grant #P40RR017447). According to the product specification sheet, human bone marrow aspirate was drawn and mononuclear cells were separated using density centrifugation. The cells were plated to obtain adherent human marrow stromal cells, which were harvested when cells reached 60%-80% confluence. These specimens were considered passage zero (P0) cells. These P0 cells were expanded, harvested, and frozen at passage 1 (P1) for distribution. Prior to release, two trials of the frozen P1 cells were analyzed over three passages for Colony Forming Units, cell growth, and differentiation into fat, bone and chondrocytes (at P2 only). These characterized hMSCs from P1 were expanded and used for neural induction protocols.

For expansion of cells from the initial frozen vial, 1×10⁶ million cells were plated onto a T75 flask and incubated overnight at 37° C. and 5.0% CO₂ for 24 hours. The media used for culture contains MEM Alpha, penicillin/streptomycin, glutamine, and FBS Select (Atlanta Biologicals). Cells were then released using Trypsin/EDTA 0.25% (Stem Cell Technologies) and incubated for 5 minutes. Trypsin was inactivated with culture medium containing 16.5% FBS. Cells were centrifuged at 1500 RPM for 5 minutes to pellet them. Supernatant was removed and the cells were washed in culture medium and centrifuged once more to remove residual Trypsin/EDTA. Cells were plated at approximately 10,000 cells per T75 flask and incubated at 37° C. and 5.0% CO₂. At approximately 80-90% confluency the process was repeated in order for the cells to begin the next passage.

B. Neural induction. For neural induction of hMSCs, several protocols were tested. These included simultaneous exposure of hMSCs to epigenetic modifiers, in particular, inhibitors of DNA methylation and histone deacetylation and to a neural environment (NSC-conditioned medium and fixed NSCs) or neural induction factors (neurotrophins, mitogens, sonic hedgehog, retinoic acid, ascorbic acid, and pharmacological agents that increase intracellular cAMP levels). Human MSC cultures manipulated according to these different protocols were fixed with paraformaldehyde at 24 h and at 1, 2, 3, and 4 weeks after treatment, followed by staining for several neural markers, such as Nestin, Sox-2, A2B5, NCAM, GFAP, B3T, MAP2, and NeuN. Thus, the initial screening test for neural differentiation was based on immunocytochemistry and morphology.
C. Immunocytochemistry. For immunocytochemistry, cells were permeabilized (5 min with 1% BSA and 0.5% Triton X-100 in PBS) and incubated for one hour with one of the following primary antibodies in the same solution (except for 10-fold less Triton X-100):

monoclonal anti-nestin (1:500, Chemicon)
polyclonal anti-Sox2 (1:800, Chemicon)
monoclonal anti-A2B5 (1:200, Chemicon)
polyclonal ant-NCAM (1:500, Millipore)
polyclonal anti-GFAP (1:1000, Novus Biologicals)
monoclonal anti-GFAP (1:400, Chemicon)
polyclonal anti-b-III-tubulin (B3T) (1:2500; Covance)
monoclonal anti-b-III-tubulin (B3T) (1:750; Covance)
monoclonal MAP2 (1:1000, Abcam)
polyclonal MAP2 (1:500, Millipore)
monoclonal NeuN (1:200, Millipore)
monoclonal anti-fibronectin (1:400, Sigma)
monoclonal anti-vimentin (1:100, StemGent)

Immunoreactive cells were visualized with AMCA-conjugated goat anti-mouse, Texas Red (TxR)-conjugated goat anti-mouse IgG, or fluorescent-conjugated (FITC) goat anti-rabbit IgG (Jackson ImmunoResearch Laboratories, Inc., West Grove, Pa.). In some experiments, FITC-conjugated goat anti-mouse IgG and TxR-conjugated goat anti-rabbit IgG were used. Glass coverslips were then mounted in ProLong Antifade reagent (Molecular Probes) to retard fluorescence quenching and dried on microscope slides. A Nikon inverted microscope equipped with color digital camera (Spot II) was used to capture representative images. Metamorph software (Universal Imaging) was used for cell counts. In the Results section, below, values represent the average from two different experiments with standard deviations.

Further neural differentiation studies were accomplished through neural induction experiments in which MSCs expressed neural stem cell, neural progenitor and mature neural markers and at the same time exhibited neuronal and glial morphology. Western blot, RT-PCR and ELISA methods were used for further characterization of NI-hMSCs.

D. Western blotting. Neural-like hMSCs grown in 6-well tissue culture plates were used for preparation of whole cell extracts. All media was removed, rinsed with PBS, and the cells were incubated in 1 ml/well trypsin/EDTA (Invitrogen) for 5 min at 37° C. Cold, sterile 1×PBS (1 ml/well) was added to each well after incubation. The suspension was centrifuged at 500 g for 2 min. The supernatant was discarded, and the cells were resuspended in 60-120 μL lysis buffer (50 mM Tris HCl, 1% SDS, 1 mM EGTA, 1 mM EDTA, 1% IGPAL, 5 μl/ml PMSF, 10 μl/ml protease inhibitor cocktail). After boiling for 5 min, the lysed cells were centrifuged for 30 s in a microfuge (10,000×g) and sonicated. The insoluble material was removed by centrifugation at 10,000 g for 10 min at 4° C. Protein concentrations were determined using a BCA Protein Assay Kit (Thermo Scientific, Inc.). The sample was diluted 1:20 in working solution of the kit (50:1 solution A: B) on a 96-well plate along with a serially diluted standard curve from a BSA standard. After incubation for 30 min at 37° C., the optical density was read using a Biotek PowerWaveXS spectrophotometer at a wavelength of 550 nm and GenS software. Depending on the concentration, samples were diluted in 50-100 μL Laemmli sample buffer (Sigma) and frozen at −20° C.

Equal amounts of protein extracted from cells (20 μg) were electrophoretically resolved on 4-15% SDS-polyacrylamide gradient gels (Bio-Red, Richmond, Calif.). For Western blotting, proteins were transferred to nitrocellulose membrane using the transfer buffer of Towbin, et al. [Proc Natl Aced Sci USA 76(9), 4350-4354 (1979)]. Equal loading was verified by staining the nitrocellulose membrane after protein transfer with Ponceau S stain. After blocking with 5% reconstituted dry milk in Tris-buffered saline and washing thoroughly between each step, blots were incubated with the primary antibodies, mouse polyclonal Sox2 (1:1000, Chemicon International, Inc., Temecula, Calif.,), polyclonal B3T (1:2500, Covance), monoclonal NeuN (1:200, Millipore), 75 KDa and 280 kDa MAP2 (1:500, Millipore). After incubation with HRP-conjugated secondary antibody (1:10000, Pierce Chemical Company, Rockford, Ill.), the protein bands were detected using the chemiluminescent substrate SuperSignal West Dura or SuperSignal West Maximum Sensitivity Substrate (Pierce Chemical Company, Rockford, Ill.) and by capturing and digitizing the images with Kodak Image Station 2000MMT.

E. RNA Extraction and Quantitative Real-time RT-PCR Analysis. Total cellular RNA was extracted from untreated hMSCs and NI-hMSCs (at 24 h, 1, 2, 3 weeks) by using the PureZOL RNA Isolation Reagent and the Aurum Total RNA Mini Kit (Bio-Rad, Hercules, Calif.). A cDNA was synthesized from 1 ug of total RNA using reverse transcriptase (iScript cDNA Synthesis Kit, Bio-Rad, Hercules, Calif.). For quantitative RT-PCR, real-time polymerase chain reaction was conducted on a Bio-Rad iCycler. Genes amplified by 0.5 umol of both sense and antisense primers and amplification was monitored using iQ SYBR Green Supermix (Bio-Rad). Forward and reverse primer sequences were designed to each of the following genes using publicly available sequence information:

| Genes | PCR fragment size (bp) |
|---|---|
| cMYC | 159 |
| NANOG | 154 |
| OCT4 | 142 |
| KLF4 | 105 |
| SOX2 | 126 |
| NCAM | 178 |
| b-ACT | 234 |
| MAP2 | 367 |
| BIIIT | 317 |
| NEFM | 312 |

To check whether amplification yields PCR products with a single molecular weight, the PCR products were electrophoresed on 2% agarose gels containing ethidium bromide. A melting curve analysis was performed after the amplification phase to eliminate the possibility of non-specific amplification or primer-dimer formation. Analysis of relative gene expression was conducted by 2^{-ΔΔC}T method [Livak and Schmittgen, Methods (San Diego, Calif. 25(4), 402-408 (2001)].
F. NGF, NT-3, GDNF, and BDNF Emax Immunoassay System. To study whether differentiated NI-hMSCs release neurotrophic factors that are important for neuronal survival and growth, an Emax immunoassay system for NGF, NT-3, GDNF and BDNF was used according to the manufacturer's protocol provided (Promega Corporation, Madison, Wis.). The Emax immunoassay system is designed for the sensitive and specific detection of neurotrophic factors such as NGF, NT-3, GDNF, and BDNF in an antibody sandwich format.

For these studies flat-bottom 96-well plates were coated with polyclonal antibody (pAb) against one of soluble neurotrophic factors NGF, NT-3, GDNF, or BDNF. Samples (100u1) from differentiated NI-hMSCs that grown for 24 h prior to immunoassay in defined medium such as Neurobasal A/B27 were added in antibody-coated wells. Next, the captured neurotrophic factors were bound by a second specific monoclonal antibody (mAb). After washing, the amount of specifically bound mAbs was detected using a species-specific antibody conjugated to horseradish peroxidase (HRP) as a tertiary reactant. The unbound conjugates were removed by washing, and samples incubated with a chromogenic substrate. The color change was measured at 450 nm on the plate reader within 30 minutes of stopping the reaction. The NGF, NT-3, GDNF, and BDNF standards provided with this system were used for generation a linear standard curve from 7.8-500 pg/ml. Data represented the means of two independent experiments performed in duplicate. Control tests did not indicate any neurotrophins in Neurobasal A/B27.

### 3. Results

Human MSCs frozen at passage P1-P6 were used for all studies. According to the product specification sheet, hMSCs from P1 (FIG. I.a) were tested for differentiation into fat, bone (FIG. I.b, 1.c), and chondrocytes (at P2 only). For induction of these characterized hMSCs into neural phenotypes, several protocols were tested (see Experimental Procedures section, above). The initial immunocytochemistry and morphology screening tests demonstrated that the experiments in which cells were treated simultaneously with inhibitors of DNA methylation, histone deacetylation, and pharmacological agents that increased cAMP levels was very effective in producing cells with neural morphology and a neural gene expression profile. In these experiments, Trichostatin A (TSA) was used as an inhibitor of histone deacetylation. Pharmacological agent 5-aza-2′-deoxycytidine (5azadc) (a chemical agent that incorporates into DNA during DNA synthesis) and RG-108 (also known as N-phthalyl-1-tryptophan, a pharmacological agent that inhibits DNA methyltransferase; see, e.g., U.S. patent application publication no. 20110207692) were used as DNA methylation inhibitors. BrcAMP (hydrolysis-resistant form of cAMP), Forskolin (adenylate cyclase activator), and IBMX or Rolipram (inhibitors of cAMP phosphodiesterases) were used as elevators of intracellular cAMP levels.

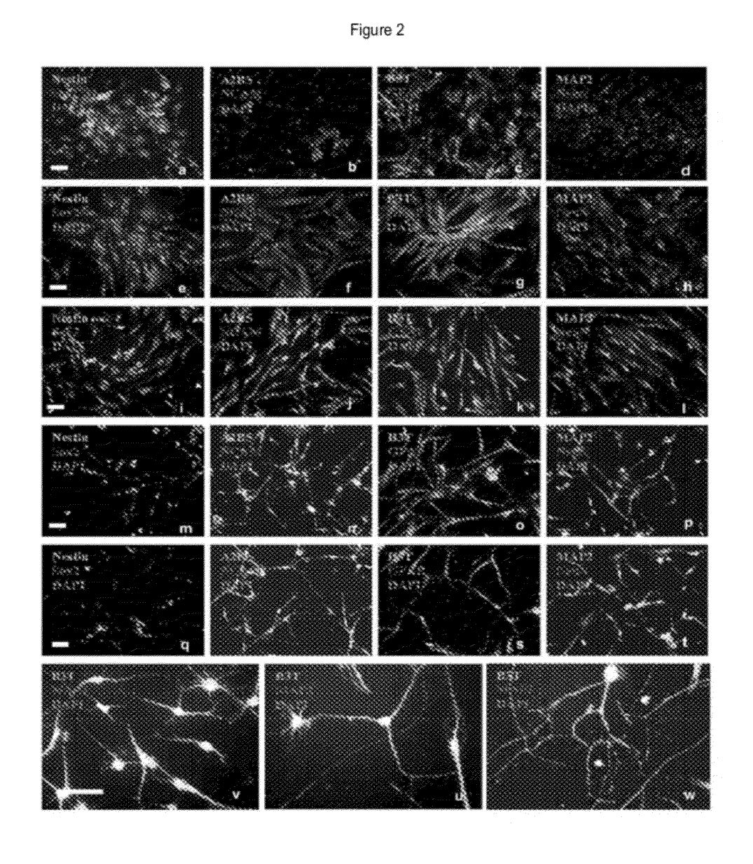

One of the most efficient methods for neural induction discovered during these studies was the combination treatment where hMSCs were exposed to 200 nM TSA, 3 mM RG-108, 300 mM 8-BrcAMP, and 1 mM Rolipram, in the medium of NSA/N2 supplemented with 20 ng bFGF. This treatment induced gradual neural changes in morphology and the gene expression profile (FIG. 2). To understand the mechanisms that underlie reprogramming of MSCs into neural cells, the expression of several genes responsible for cell pluripotency and neural differentiation was studied by using Western blot, real time RT-PCR, and immunocytochemistry techniques.

Immunocytochemical results showed that, similar to untreated hMSCs (FIG. 2.a-2.d), hMSCs grown in this particular neural induction condition for 24 h barely expressed the neural markers nestin, A2B5, NCAM, and NeuN (FIG. 2e-2h). In 24-treated cultures, 2%±1.15% of cells were positive to nestin, which was approximately the same as for non-treated hMSCs. The percentage of cells highly positive to B3T and Sox2 was also very small (approximately 2%). However, 20%-30% exhibited low immunoreactivity to these markers. The percentage of cells positive to neural progenitor and mature neural markers Sox2, nestin, A2B5, NCAM, B3T, GFAP, NeuN, and MAP2 gradually increased during the next two weeks of treatment (FIG. 2.i-2.p); and at the end of week three, they had increased 15%±11.9, 14%±10.63, 77.6%±10, 49%±22, 57%±17.2, 17%±7.7, 28.5%±6.2, and 44.1%±7.5, respectively (FIG. II.q-t). In total, 95% of cells were positive to neural markers. Twenty to thirty percent of cells also produced long axon- and dendrite-like extensions (FIG. 2.v-2.w). Only a small percentage (2-4%) of cells with fibroblastic morphology were positive to fibronectin and (5-7%) to vimentin.

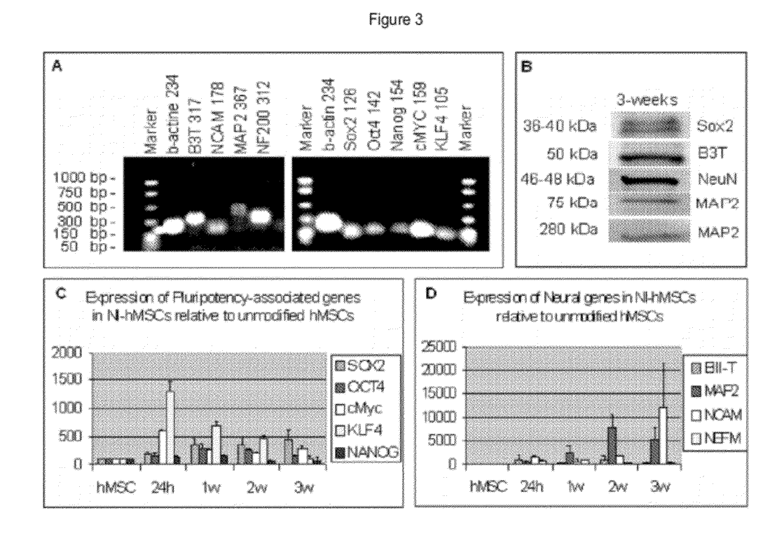

Expression of neural markers (50 kDa B3T, 70 kDa low-molecular weight MAP2, 280 kDa high-molecular weight MAP2, NeuN 46-48 kDa range, and 36-40 KDa Sox-2) in untreated hMSCs and differentiated NI-hMSCs was examined by Western blotting (FIG. 3.A). The results showed that while untreated hMSCs expressed neural markers such as Sox-2 and B3T, the expression levels were lower than in 3-week treated NI-hMSCs. Fold increases of Sox2 and B3T expression (analysed by densitometry) were 4.2±2.3 and 2.1±1.5, respectively. In addition, NI-HMSCs expressed several other neuronal markers such as a low-molecular weight MAP2, a high-molecular weight MAP2, and NeuN (FIG. 3.A).

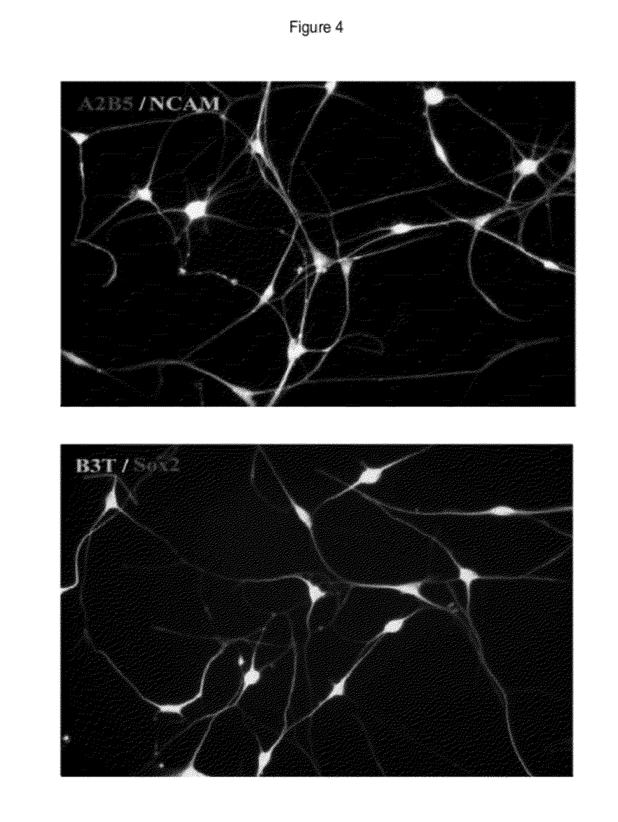

RT-PCR studies demonstrated that, in 24 h-treated cultures, the expression level of the pluripotency-associated genes Oct-4, Nanog, Klf4, c-Myc, and Sox-2 were increased in comparison to non-treated MSCs (FIG. 3.B). During the next three weeks, the expression of Oct-4, KLf4, and Nanog gradually decreased. The expression level of Sox2, which can be considered as a marker for pluripotency as well as a marker for neural cells, gradually increased during the next three weeks of treatment (FIG. 3.B). In contrast to pluripotent genes, expression levels of neural genes were gradually increased during the 3 weeks of treatment (FIG. 3.D). Expression of B3T, which was highly expressed even in untreated hMSCs (FIG. 3A) slightly fluctuated during the 3 weeks of treatment (FIG. 3.D). The expression of neural markers NEFM, MAP2, NCAM, and Sox2 in NI-hMSCs was significantly different form hMSCs (P<0.05) for 1-week, 2-week, and 3-week treated samples, respectively. The results of agarose gel electrophoresis revealed bands of the expected sizes for all PCR products (FIG. 4.C) for 1-, 2-, and 3-week studies.

Next, whether differentiated NI-hMSCs released neurotrophic factors important for neuronal survival and growth was studied. To this end, culture media of differentiated (2-week) NI-hMSCs were changed to Neurobasal-A 24 h before neurotrophin release measurement studies. Using NGF, BDNF, NT3, and GDNF, the Emax immunoassay system (Promega) demonstrated that NI-hMSCs released neurotrophic factors GDNF (157±9 pg/ml//1×10⁵/day) and BDNF (5.3±3 pg/ml//1×10⁵/day). Dopamine was also shown to have been released by these cells.

### 4. Discussion

Although differentiation was once thought to be an end-point in development, accumulating evidence indicates that the differentiation state of somatic nucleus remains sufficiently plastic such that it may be reprogrammed. As is known, ectopic expression of a defined set of factors, such as Oct-4, Sox2, KLF4, and c-Myc (or LIN28 instead of cMyc) can convert terminally differentiated skin cells into pluripotent embryonic stem cells [Takahashi and Yamanaka, Cell 126(4), 663-676 (2006); Okita, et al., Nature 448(7151), 313-317 (2007); Yu, et al., Science 318(5858), 1917-1920 (2007); Takahashi, et al., Cell 131(5), 861-872 (2007); Eggenschwiler and Cantz, Hepatology 49(3), 1048-1049 (2009); Okita, et al., Science 322(5903), 949-953 (2008); Zhou, et al., Cell stem cell 4(5), 381-384 (2009). Interestingly, some recent reports have suggested that somatic cells can also be reprogrammed by inducing ectopic expression of only two factors, such as Oct4 and KLF4 or Oct4 and c-Myc [Kim, et al., Nature 454(7204), 646-650 (2008)] or just simply by manipulating environmental conditions [Page, et al., Cloning and stem cells 11(3), 417-426 (2009)].

Several other recent studies demonstrated that cells could also be reprogrammed with small molecules, particularly by those that are involved in the regulation of chromatin structure. In one of these studies, Lyssiotis, et al. showed that global histone acetylation, induced by inhibitor of HDAC, can partially reverse the lineage restriction of oligodendrocyte precursor cells, thereby inducing developmental plasticity [Proc Natl Aced Sci USA 104(38), 14982-14987 (2007)]. In another study, hematopoietic cells were generated by transient epigenetic modification of neurospheres by the chromatin-modifying agents TSA and 5-azadc [Schmittwolf, et al., The EMBO journal 24(3), 554-566 (2005)]. Global gene expression analysis performed by the same group revealed that TSA/5azadc treatment induces transient expression of several stem cell and pluripotency-associated genes [Ruau, et al., Stem Cells 26(4), 920-926 (2008)].

The experiments described in this example confirm that transient expression of pluripotency-associated genes can be a triggering factor for high cellular plasticity and transdifferentiation. Indeed, these experiments show that neural-like cells can be produced from BM-derived hMSCs by simultaneously exposing cells to chromatin-modifying agents and neural inducing factors. As demonstrated in the Results section, one of the most efficient methods for neural induction was a combination treatment where hMSCs were exposed to epigenetic modifiers (inhibitors of DNA methylation and histone deacetylation) and pharmacological agents that increased cytosolic cAMP levels. Gene analysis showed a significant initial increase in pluripotency-associated gene expression, which declined during the next 2-3 weeks, followed by increases in the expression of neural-associated genes. This gradual increase of neural gene expression accompanied neural morphological changes. Neural induction of MSCs elevating camp levels only transiently upregulates neural markers rather than inducing neural differentiation. Together, these results indicate that dedifferentiation of adult stem cells into to a less differentiated (although not necessarily pluripotent) state by epigenetic modifiers is an initial necessary step before inducing the cells into different lineage.

### 5. Conclusions

Human (or other mammalian) MSCs neurally modified in accordance with reprogramming and neural induction methods of the invention exhibit several neural characteristics, as assessed by morphology, gene expression profiles, immunocytochemistry, and ability to release neurotrophic factors such as GDNF, BDNF, and dopamine in vitro. Indeed, 75-95% of cells produced by such methods are positive for neuronal progenitor and mature neuronal markers. Indeed, neural progenitors, for example, A2B5 and NCAM positive cells, can be sorted (e.g., by FACS) based on surface markers and differentiated to appropriate neural cells. NI-hMSCs produced in accordance with the invention also are responsive to neurotrophic factors. For example, the expression level of the cholinergic marker ChAT was increased by 2-3 fold when NI-hMSCs were grown for 1 week with BDNF and GDNF.

Two weeks post-treatment, NI-hMSC cultures were found to consist mostly of neural progenitors and partially differentiated cells. This makes such cell populations ideal for CNS cell replacement therapy since neuronal- and glial-committed cells (relatively more differentiated cell types) are much more likely to differentiate and produce mature neural cells in vivo. In addition, these neurally modified cells could be useful therapeutic tools for CNS disorders since these cells secret dopmanine and neurotropic factors such as BDNF and GDNF.

The experiments described in this example thus indicate that neurally-induced hMSCs may provide an suitable source of allogeneic, autologous, or syngeneic adult stem cells that can be used for replacing damaged neural cells in patients or subjects having CNS injury or disease and/or to provide support to CNS tissue. In particular, these results show:
a. an efficient method for generation of neural-like cells from adult human bone marrow derived mesenchymal stem cells has been developed;
b. the importance of reactivating pluripotency-associated genes in MSCs before or during exposure to neural inducing factors;
c. neural induction can be achieved by exposing cells simultaneously to inhibitors of DNA methylation and histone deacetylation and one or more pharmacological agents that increase cAMP levels;
d. confirmation of expression of pluripotency and neural markers by immunocytochemistry, Western blot, and RT-PCR;
e. NI-hMSCs cells released the neurotrophic factors GDNF and BDNF, a determined by ELISA; and
f. after three weeks of treatment, 95% of cells were positive to neural markers and 20-30% percent of cells produced long axon- and dendrite-like extensions.

### Example 2

### Transplanted Neurally Modified Human Bone Marrow Derived Mesenchymal Stem Cells Promote Tissue Protection and Locomotor Recovery in Spinal Cord Injured Rats

### 1. Introduction

Stem cell-based therapy to repair and replace lost neural cells is a highly promising treatment modality for central nervous system (CNS) disease. Mesenchymal stem cell, particularly bone marrow (BM)-derived mesenchymal stem cells (MSCs), have great potential as therapeutic agents against neurological maladies since they have the ability to differentiate into neural phenotypes and can be readily isolated and expanded for allogenic, autologous, or syngeneic transplantation with little or no risk of rejection. Example 1, above, described a new method for efficient generation of neural-like cells from human BM-derived MSCs (hMSC). Neural induction was achieved by exposing cells simultaneously to inhibitors of DNA methylation, histone deacetylation, and pharmacological agents that increased cAMP levels.

The aim of the study described in this example was to determine whether transplanted neurally induced hMSCs (NI-hMSCs) could survive, differentiate, and promote tissue protection and functional recovery in injured spinal cord (ISC) rats. To this end, three groups of adult female Sprague Dawley rats received PBS,

MSCs, or NI-hMSCs, one week after injury. Functional outcome was measured using the BBB scale and a thermal sensitivity test on a weekly basis up to 12 weeks post-injury. Graft integration and spinal cord anatomy was assessed by stereological, histochemical, and immunohistochemical techniques. Results demonstrated that transplanted NI-hMSCs survived, differentiated, and significantly improved locomotor recovery of ISC rats. Transplantation also reduced the lesion cavity volume and promoted white matter sparing as compared to the controls. Thus, hMSCs neurally induced in accordance with the invention could provide an alternative source of adult stem cells that could be useful for injured or diseased CNS regeneration.

### 2. Methods

A. Expansion of hMSCs. Human bone marrow MSCs were expanded as described in Example 1, above.
B. Neural induction. Neural induction was performed by exposing the hMSCs to 200 nM trichostatin A (TSA) (a histone deacetylase inhibitor), 30 uM RG-108 (a DNA methyltransferase inhibitor), 300 uM 8-BrcAMP (a highly stable, biologically active form of cAMP), and 1 uM Rolipram (inhibitor of phosphodiesterases), in the medium of NeuroCult/N2 supplemented with 20 ng bFGF. After two weeks of treatment cells were used for transplantation.
C. Spinal cord injury and transplantation procedures. All animal surgeries were performed in accordance with animal surgery guidelines established by the Medical College of Wisconsin and Zablocki Veterans Affairs Medical Center Veterinary Medical Unit. For these studies, Sprague-Dawley female rats (200-250 g body weight) were anesthetized using intraperitoneal ketamine (75 mg/kg) and medetomidine (0.5 mg/kg). Rats were placed prone on an operating table covered with a warming blanket. The dorsal mid-thoracic region was shaved and prepped with Betadine. Using sterile technique, an incision was made over the mid-thoracic region and a subperiosteal dissection was performed. Three spinal-level laminectomies (T7-9) exposed the underlying spinal cord. Hemostasis was obtained with Surgical Gelfoam, and bone edge waxing. An NYU Impactor was used to produce a consistent, uniformly sever injury (10-g drop from a height of 25 mm directly onto the dura at level T8). Following impact, the wound was closed in layers. Post-operatively, all animals were given one dose of enrofloxacin (10 mg/kg subcutaneously). Subcutaneous lactated Ringer's solution (12 ml) was provided and made available as needed (50 ml/kg). Bladder expression was performed manually twice per day until the animals were able to void independently. One week after SCI, adult female Sprague Dawley rats were randomized to receive NI-hMSCs, hMSCs, or PBS. Ten animals from each group were for behavioral studies and ten for stereological and immunohistochemical studies. For the transplant surgery, rats were re-anesthetized and, using sterile technique, the SCl site was re-exposed. NI-hMSCs were loaded into a 25-μl Hamilton syringe at a concentration of 100,000 cells/10 μl. Under microscopic visualization, the cells were stereotactically injected into the spinal cord on either side of midline, 1 mm rostral and 1 mm caudal to the injury site. Four 2.5-μl injections delivered a total of 100,000 NI-hMSCs to each spinal cord. After injection, the surgical site was closed in multiple layers and the animals were allowed to recover with analgesia and post-operative care, as described above. All animals received the immunosuppressant Prograf (50 mg/kg) on a daily basis. All groups received Prograf in order to account for any neuroprotective effects of the immunosuppressant.
D. Behavioural testing. Individuals involved in assessing functional recovery of the animal subjects were blinded with regard to treatment. Locomotor function was evaluated using the BBB Locomotor Recovery Scale for open-field walking [Basso, et al., J. Neurotrauma. 1995 February; 12(1):1-21]. Behavioural tests for thermal allodynia were performed before and after SCl in both forelimbs and hindlimbs, as previously described [Bennett, et al. Pain. 2000 May; 86(1-2):163-75; Hains, et al., Neuroscience. 2003; 116(4)1097-110], The response to thermal stimulation was measured by latency of forelimb and hindlimb paw withdrawal to radiant heat of 55° C. Briefly, rats were placed in a Plexiglas cylindrical container over a radiant heat source and latency to response was recorded. An average of three trials was recorded and non-responders were removed from the hot plate after 60 sec. Brisk paw withdrawal with or without accompanying supraspinal reflexes such as head turning, paw guarding, licking, biting, or vocalization, were considered positive responses to the thermal behavioral testing. In all groups, behavioral scoring was performed prior to injury, after injury, prior to transplantation, and then weekly for 12 weeks post-transplantation. Effects of treatment were assessed using two-way ANOVA followed by posthoc Tukey's analysis with significance level of p<0.05.
E. Immunohistochemistry and histological assessment. After 24 h and 1, 2, 4, and 12 weeks animals were given an overdose of Nembutal and perfused intracardially with 0.9% PBS followed by 4% paraformaldehyde in PBS. Spinal cords were dissected and the injury site at T8 identified. The cervical C6-T1 region was identified by the origin of the median-ulnar forelimb nerve complex from the spinal cord at C7-T1 vertebral levels. The spinal cord was cut into segments that included the T8 region with 2-mm uninjured cord rostral and caudal to the injury, and the cervical cord C6-T1. These segments were post-fixed in 4% paraformaldehyde/PBS at 4° C. for 1 h and cryoprotected in 30% sucrose/PBS overnight at 4° C. To eliminate or reduce auto-fluorescence, two reagents, sodium borohydride and Sudan Black B (Sigma) were used. To this end, post-fixed cryostat cross-sectioned samples immersed in ice cold PBS supplemented with 1 mg/ml sodium borohydride for 40 min. After this, samples were washed in PBS, permeabilized 5 min with 1% BSA, 2% goat serum, and 0.5% Triton X-100 in CMF-PBS, and incubated for 1 hour with one of the following primary antibodies in the same solution (except for 10 fold less Triton X-100): human anti-mitochondrial antibody (monoclonal, 1:400, Millipore), polyclonal anti-Sox2 (1:800, Chemicon); polyclonal anti-GFAP (1:1000, Novus Biologicals, Inc., Littleton, Colo.); and polyclonal anti-B3T (1:1000, Covance, Princeton, N.J.). After 3 washes of 5 min in PBS, the sections were incubated in PBS containing 1% BSA, 2% goat serum, and one of the following secondary antibodies: Texas Red-conjugated (TxR) goat anti-mouse IgG and FITC-conjugated goat anti-rabbit. Nuclear counter-staining was achieved with DAPI or Topro. Cells were then treated with Sudan Black (0.3% in 70% ethanol) for 1 min, rinsed in PBS and coverslipped with Fluoromount G mounting medium (Electron Microscopy Sciences, Hatfield, PA). A Nikon inverted microscope equipped with color digital camera Spot II (Diagnostic Instruments, Inc., Sterling Hts., MI) or BioRad confocal microscope were used to capture representative images. Metamorph software (Universal Imaging, Downingtown, PA) were used for cell counts.

For histological assessments of white matter sparing 20 um transverse spinal cord sections of rats that were sacrificed at 12 weeks after SCl were stained with eriochrome cyanine (to stain myelinated white matter), and Eosin/Phloxin (to visualize cells and gray matter). Stained sections were viewed on an optical microscope to ensure that all samples contained the entire lesion extent and to identify injury epicenter. The section containing injury epicenter was defined visually as the one with a smallest visible rim of spared myelin and with a largest cystic cavity. The eriochrome cyanine-positive area (white matter area) was assessed by using Metamorph color threshold tool. The percentage of spared white matter in each section was calculated by dividing the white matter area by total cross-sectional area and multiplying by 100. The percentages of spared white matter for 13 evenly-spaced sections 1 mm apart (picked up from approximately 3 mm rostral and caudal to the lesion epicentre) were summed and means and standard errors calculated for each treatment group (PBS, hMSCs, NI-hMSCs). One-way ANOVA and a Fisher's LSD post hoc were used to determine significant differences between groups. To assess the volume of cystic cavities 13 transverse spinal cord sections with an equal distance (500 μm) spanning ±3 mm from epicenter were used. The total estimated volume was calculated using the Cavalieri's Principle. The individual subvolumes were obtained by multiplying the cavity area by the distance between sections, and the subvolumes were summed to generate the total volume of cystic cavities (Σn [cystic cavities areas×intersection distance], n=number of sections analyzed). Lesion cavity volumes expressed as a percentage of the volume of spinal cord T8 segment (3 mm rostral and caudal to the lesion epicenter) that calculated by dividing the cystic cavity volume by the spinal cord T8 segment volume and multiplying by 100. For statistical analysis, the group means were compared with one-way ANOVA and Fisher's LSD post hoc. Mimics 8.11 3D cord modeling software was used for three-dimensional reconstruction of lesion cavities.

### 3. Results

Human MSCs grown in neural induction medium for two weeks produced cells that were positive to several neural stem cell, neural progenitor, and mature neuronal markers (FIG. 4). At this stage NI-hMSCs were used for transplantation studies.

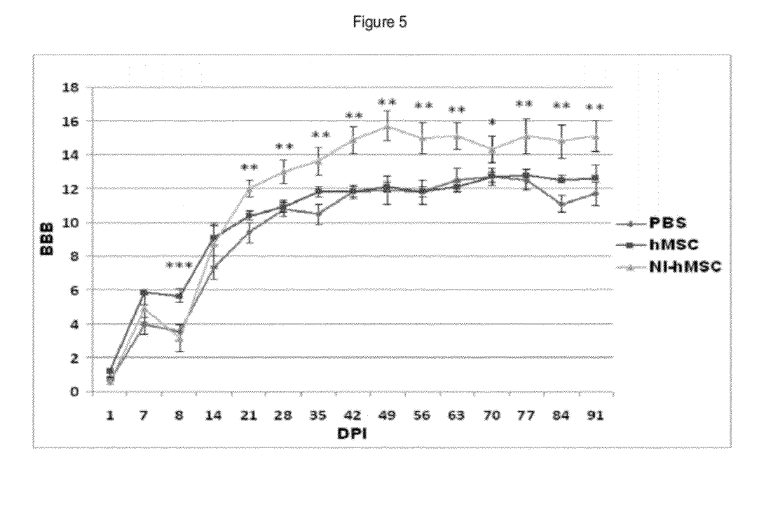

To study the therapeutic effect of NI-hMSCs on locomotor and sensory functions after SCl, cells were transplanted into the moderately injured spinal cord of rats. The control groups were injected with hMSCs and PBS at the same volume. The BBB Locomotor Recovery Scale was used during open-field walking observations to evaluate locomotor function. Assigned BBB scores reflected the near complete loss of hindlimb motor function that was observed in all groups by one week after injury, and also prior to transplantation with NI-hMSCs, hMSCs, and PBS. After the intraspinal transplantation procedure, hindlimb function improved gradually in all groups irrespective of what used for transplantation. Motor recovery that consisted of hindlimb weight support and consistent hindlimb stepping was significantly different at 2-12 weeks post-recovery in the group that was transplanted with NI-hMSCs when compared with the control groups that received hMSCs and PBS (FIG. 5).

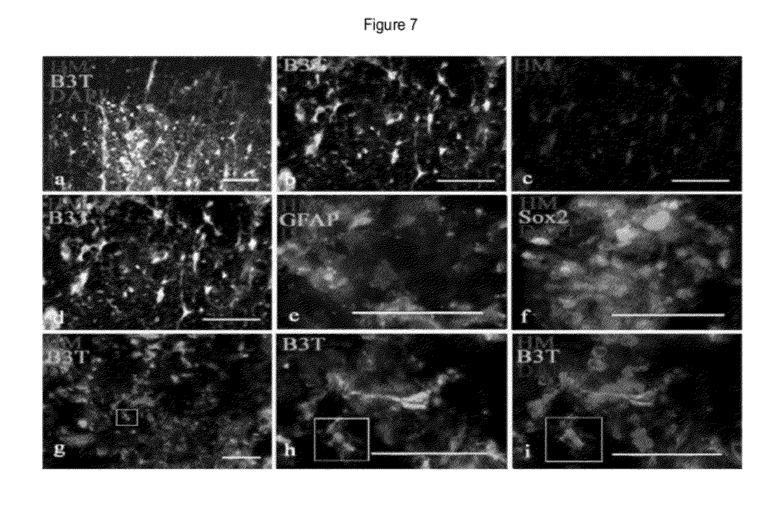

Immunohistochemistry data showed that NI-hMSCs were survived at post transplantation weeks 1-12. Analysis of the spinal cord slices of two weeks treated animals revealed that 85% percent of survived cells were positive to B3T (FIGS. 7.a, 7.b, 7.c). A small percentage of cells (2%) was positive to GFAP (FIG. 7.e). By 12 weeks the number of surviving cells declined to 15-20% of that at week 2 and only 10% of survived cells were positive to B3T (FIGS. 7.g, 7.h, 7.i).

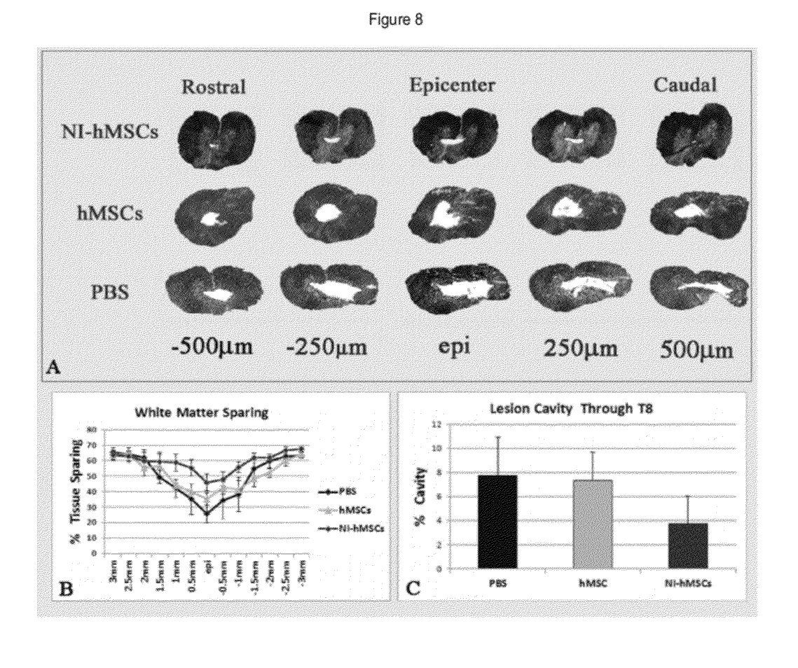

Histological studies of spinal cord sections at specified distances rostral and caudal to the epicenter demonstrated that at the epicentre and 1 mm caudal and rostral from it the percentage of the eriochrome cyanine-positive spared white matter was significantly larger in NI-hMSCs treated group than that in the PBS group (FIG. 8.A, 8.B). While there was no significant difference between naïve hMSCs and PBS groups, there was a modest trend for increased white matter sparing in hMSCs-treated versus PBS-treated spinal cords (FIG. 8.B). Stereological assessments of injured spinal cord tissues demonstrated a modest reduction in the percentage of cystic cavities in the NI-hMSCs and hMSCs treated groups versus PBS group (FIG. 8.C). Although no statistically significant difference had been noticed between groups, the difference found between NI-HMSCs and PBS was very close to the significance level adopted in the study (p<0.05).

### 4. Discussion

Stem cell transplantation therapy has recently emerged as a powerful and promising strategy for the treatment of several neurological disorders including spinal cord injury. Several kinds of cells have been considered as potential candidates for such therapies. Among them are mesenchymal stem cells since they readily can be isolated and expanded, for example, for auto- or allo-transplantation, and because they have the ability to differentiate into cells with numerous neural properties and exhibit beneficial effects in several animal models of neurological disorders including spinal cord injury. Mesenchymal stem cells (MSCs) have demonstrated a measurable therapeutic effect following transplantation into animal models of spinal cord injury and spinal cord injured patients.

Example 1, above, describes an efficient method for generating neural-like cells from bone marrow derived MSCs. This unique methodology was developed based on the inventor's understanding of stem cell plasticity. The inventivesness of this approach is simultaneous reactivation of pluripotent and neural genes by exposing MSCs simultaneously to inhibitors of DNA methylation and histone deacetylation and pharmacological agents that increase cAMP levels. Neural-like cells so generated exhibit numerous traits of neural cells and with further differentiation in appropriate conditions produce different neuronal and glial phenotypes.

Results presented in this example demonstrate that such NI-hMSCs, when transplanted, can survive, differentiate, and significantly improve locomotor recovery of ISC rats. Transplantation also reduced the cavity volume and increased spared white matter in ISC rats as compared to control animals. These results demonstrate that neurally modified hMSCs, in comparison to controls, promote tissue preservation and functional recovery in spinal cord injured animals. Thus, MSCs neurally modified in accordance with the invention can provide a ready, safe source of autologous, syngeneic, allogeneic, or unmatched adult stem cells that could be useful for replacing damaged neural cells in injured or diseased CNS and/or providing support to CNS tissue.

### Example 3

### Feline Bone Marrow-Derived Mesenchymal Stem Cells Express Several Pluripotent and Neural Markers and Easily Turn into Neural-Like Cells by Manipulation with Chromatin Modifying Agents and Neural Inducing Factors

### 1. Introduction

This example describes the production of a source of non-human neural stem cells, in particular, neural-like cells produced from feline bone marrow-derived MSCs (NI-fMSCs), that can be used in cellular replacement therapies in large animal models of neurological disorders. As described below, fMSCs exhibited a neural morphology after 48-72 h of neural induction. Immunocytochemistry, ELISA, Western blot, and RT-PCR studies revealed a higher level of expression of several pluripotent and neural genes in NI-fMSCs, the majority of which were expressed in untreated fMSCs at relatively low levels. Thus, the expression of pluripotency- and neural-associated genes in unmodified fMSCs make them more pliable for reprogramming into a neural fate by manipulation with chromatin modifying agents and neural inducing factors.

### 2. Experimental Procedures

A. Isolation of mesenchymal stem cells from feline marrow. Feline bone marrow was harvested with a needle through the tip of the greater trochanter into the medullary canal of the cat femur and collected into 1-5 volumes MEM Alpha Medium (GIBCO). After cells were pelleted by 500 g centrifugation, they were rinsed in the same medium. The resulting pellets were resuspended in MEM Alpha Medium containing 10% fetal bovine serum screened for its ability to support mesenchymal stem cell proliferation (FBS, Stem Cell Technologies, Vancouver, Canada), 100 U/ml penicillin, 100 mg/ml streptomycin and 2 mM Glutamine (Sigma). The cells were plated at density of 1×10⁹/cm² in 75 cm² plastic flasks and incubated at 37° C. in a humidified atmosphere with 5% CO'. Hematopoietic and nonadherent cells were removed by a change of medium after 48 h. Expanded cells were used for neural induction studies.
B. Neural induction. Neural induction was performed by the procedure described in Example 1, above. Briefly, fMSCs were exposed to 200 nM trichostatin A (TSA) (an inhibitor of histone deacetylases), 3 μM RG-108 (a DNA methyltransferase inhibitor), 300 μM 8-BrcAMP (a highly stable, biologically active form of cAMP), and 1 μM Rolipram (a phosphodiesterase inhibitor), in the medium of NeuroCult/N2 supplemented with 20 ng bFGF.
C. Immunocytochemistry. For immunocytochemistry, fMSCs and 24 h, 48 h, 72 h-treated NI-fMSCs were fixed with 4% paraformaldehyde and stained for several immature and mature neural markers, such as A2B5, NCAM, B3T, MAP2, NeuN, neurofilament 200 (NF), Nurr1 (an early marker for dopaminergic neurons), tyrosine hydroxylase (TH; a dopaminergic marker), choline acetyltransferase (ChAT; a cholinergic marker), GABAergic (GABA), and serotonin (5HT; a serotonergic neuronal marker). First, cells were permeabilized 10 min with 0.2% Triton X-100 in PBS, followed by blocking with 5% Goat serum in PBS for 30 min, then incubated for one hour with one of the following primary antibodies in PBS. The antibodies used were: mouse monoclonal anti-A2B5 (1:200, Chemicon), rabbit polyclonal ant-NCAM (1:500, Millipore), mouse monoclonal anti-β-III-tubulin (B3T) (1:750, Covance), rabbit polyclonal anti-MAP2 (1:500, Millipore), mouse monoclonal anti-NeuN (1:200, Millipore), rabbit polyclonal anti-neurofilament 200 (1:1000, Millipore), rabbit polyclonal anti-Nurr1/NOT1 (1:300, Millipore), rabbit polyclonal anti-tyrosine hydroxylase (1:150, Millipore), Rabbit polyclonal anti-ChAT (1:500, Millipore), mouse monoclonal anti-GABA (1:200, Millipore), and mouse monoclonal anti-serotonin (5HT, 1:50, Abcam). Immunoreactive cells were visualized with Texas Red (TxR)-conjugated goat anti-rabbit IgG or fluorescent-conjugated (FITC) goat anti-mouse IgG (Jackson ImmunoResearch Laboratories, Inc., West Grove, Pa.). To reduce fluorescence quenching, glass coverslips were mounted in ProLong Antifade reagent (Molecular Probes) and dried on microscope slides. Representative images were captured by a Nikon inverted microscope equipped with color digital camera (Spot II). Metamorph software (Universal Imaging) was for analyzing the images and counting the cells. In the results section, values represent the average from two different experiments with standard deviations
D. Western blotting. Cells grown in 6-well tissue culture plates were used for preparation of whole cell extracts. After removing the media, cells were rinsed twice with PBS and incubated in 1 ml/well trypsin/EDTA (Invitrogen) for 5 min at 37° C. Cold, sterile 1×PBS (1 ml/well) was added to each well after incubation. The suspension was centrifuged for 5 min at 500 g. After cells were resuspended in 60-120 μL, lysis buffer (50 mM Tris HCl, 1 mM EGTA, 1% SDS, 1 mM EDTA, 5 μl/ml PMSF, 1% IGPAL, 10 μl/ml protease inhibitor cocktail) was added and the cell suspensions were then kept on ice for 30 min. The cell suspensions were then centrifuged at 16,000 g for 15 min at 4° C. and the supernatants were collected. Protein concentrations were determined by BCA Protein Assay Kit (Thermo Scientific, Inc.). The samples were frozen at −80° C. For SDS-polyacrylamide gel electrophoresis equal amounts of protein extracted from cells (10 pg) were resolved on 4-15% polyacrylamide gradient (Bio-Red, Richmond, Calif.). For Western blotting, proteins were transferred to nitrocellulose membrane by Trans-Blot SD Electrophoretic Transfer Cell. After blocking with 5% dry milk reconstituted in Tris-buffered saline, blots were incubated with the following primary antibodies: Rabbit Polyclonal Anti-Sox2 (1:1000, Chemicon International, Inc., Temecula, Calif.,), Chichen Polyclonal Anti-c-myc (1:6000, Millipore), Rabbit Polyclonal Anti-Nanog (1:2000, Millipore), Mouse Monoclonal anti-B3T (1:3000, Covance), Mouse Monoclonal Anti-NeuN (1:1000, Millipore), Mouse Monoclonal Anti-Neuron Specific Enolase (NSE) (1:1000, Millipore), Rabbit Polyclonal Anti-Nurr1/NOT (1:1000, Millipore), Rabbit Polyclonal Anti-TH (1:800, Millipore), Rabbit Polyclonal anti-ChAT (1:1500, Millipore), and Mouse Monoclonal Anti-β-Actin (1:7000, Millipore). After incubation with Goat Anti-Rabbit/Mouse HRP-conjugated secondary antibody (1:6000, Pierce Chemical Company, Rockford, Ill.) or Rabbit Anti-chicken HRP-conjugated secondary antibody (1:6000, Thermo Scientific), the protein bands were detected using the chemiluminescent substrate SuperSignal West Dura or SuperSignal West Maximum Sensitivity Substrate (Pierce Chemical Company, Rockford, Ill.) and by capturing and digitizing the images with Kodak Image Station 2000MMT. The experiments were carried out in duplicates and the data is shown as standard error of the mean. The statistical significance was assessed by one-way AVOVA followed by Tukey's pair comparisons. P values <0.05 were considered significant.
E. Quantitative Real-time RT-PCR Analysis. Total cellular RNA was extracted from untreated fMSCs and from 24 h, 48 h, 72 h-treated NI-fMSCs by using the PureZOL RNA Isolation Reagent and the Aurum Total RNA Mini Kit (Bio-Rad, Hercules, Calif.). Complementary DNA was synthesized from 1 ug of total RNA using reverse transcriptase (iScript cDNA Synthesis Kit, Bio-Rad, Hercules, Calif.). For quantitative RT-PCR, real-time polymerase chain reaction was conducted on a Bio-Rad iCycler. Genes amplified by 0.5 μM of both sense and antisense primers and amplification was monitored using iQ SYBR Green Supermix (Bio-Rad). To check whether amplification yielded PCR products with a single molecular weight, the PCR products were electrophoresed on 1% agarose gels containing ethidium bromide. To eliminate the possibility of non-specific amplification or primer-dimer formation a melting curve analysis was performed after the amplification phase. Relative gene expression was analyzed by 2^{-ΔΔCT} method. The experiments were carried out in triplicate and repeated twice. The statistical significance was assessed by one-way AVOVA followed by Tukey's pair comparisons. P values <0.05 were considered significant.
F. Emax Immunoassay System. To study the ability of NI-fMSCs to release neurotrophic factors important for neuronal survival and growth, an Emax immunoassay system for NGF BDNF, NT3 and GDNF was used. The immunoassays were conducted according to the product protocol (Promega Corporation, Madison, Wis.). Flat-bottom 96-well plates were coated with a polyclonal antibody (pAb) to one of the soluble neurotrophic factors GDNF, BDNF, NT-3 and NGF. Samples (100u1) from treated 72 h fMSCs that were grown for an additional 24 h prior to immunoassay in defined medium (Neurobasal A/B27) were added in antibody-coated wells. The captured neurotrophic factors were bound by secondary specific monoclonal antibodies (mAbs). The samples were then washed and specifically bound mAbs was detected using a species-specific antibody conjugated to horseradish peroxidase (HRP). The unbound conjugates were removed by washing and the samples were incubated with a chromogenic substrate. After 30 min, the reactions were stopped and the color change was measured at 450 nm on a plate reader. The NGF, BDNF, NT-3, and GDNF standards provided with the Emax system were used for generation of a linear standard curve from 7.8-500 pg/ml. Data represent the means of two independent experiments performed in duplicate.

### 3. Results and Discussion

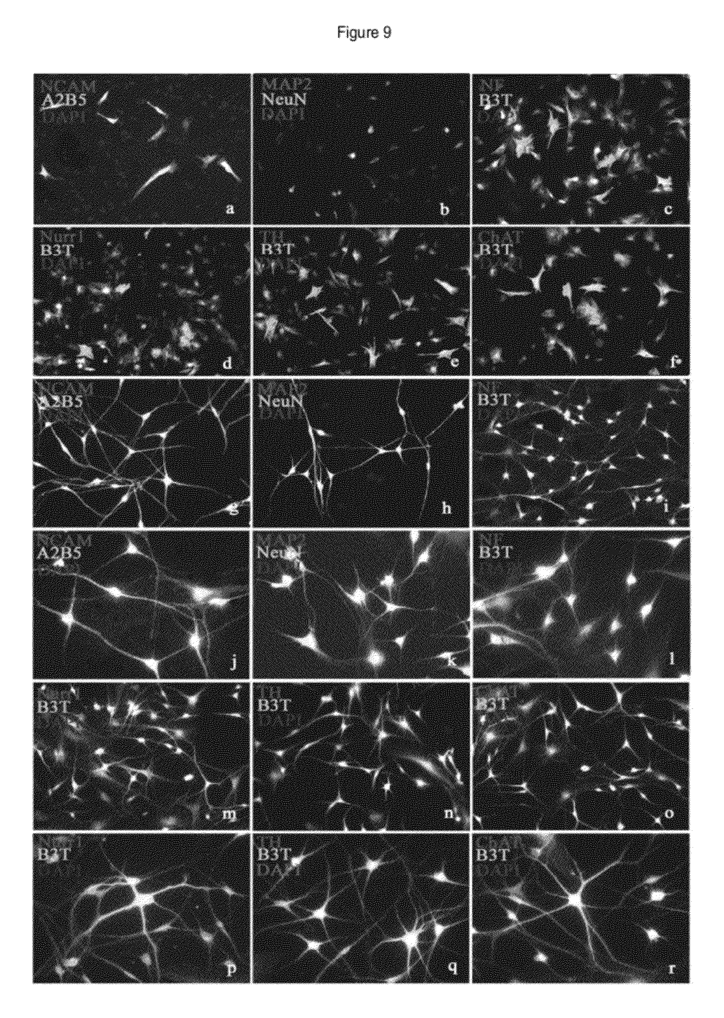

Feline MSCs grown in MEM Alpha Medium supplemented with 10% fetal bovine serum exhibited a spindle-shaped and flattened morphology. Morphologically, fMSCs appeared very similar to their rodent and human counterparts. Immunocytochemical results showed that 10%-20% of untreated fMSCs exhibited low immunoreactivity to neural markers such as B3T, NCAM, A2B5, MAP2, NeuN, NF, Nurr1, TH, and ChAT. The percentage of cells that were highly positive for B3T, NCAM, A2B5, MAP2, NeuN, and NF was 3-7% and for Nurl, TH, and ChAT was less than 0.5-1% (FIG. 9.a-4f). The percentage of cells that were positive to B3T, A2B2, NCAM, MAP2, NeuN, NF was gradually increased during the next two days during the treatment and after 72 h they were 95±11.02, 85.64%±7.85, 87.58%±9.23, 85.45%±8.45, 82.03±%, and 77.54%±8.05 respectively (FIG. 9.g-l). The percentage of cells positive for dopaminergic marker Nurr1, and TH, and cholinergic marker ChAT were 58.42%±7.51, 50.02%±8.34, and 62.34±8.45 respectively (FIG. 9.m-9.r). No cells positive for GABA and 5-TH were observed. In total, more than 95% of cells were positive to neural markers. Only a small percentage (less than 5%) of cells still retained a fibroblastic morphology.

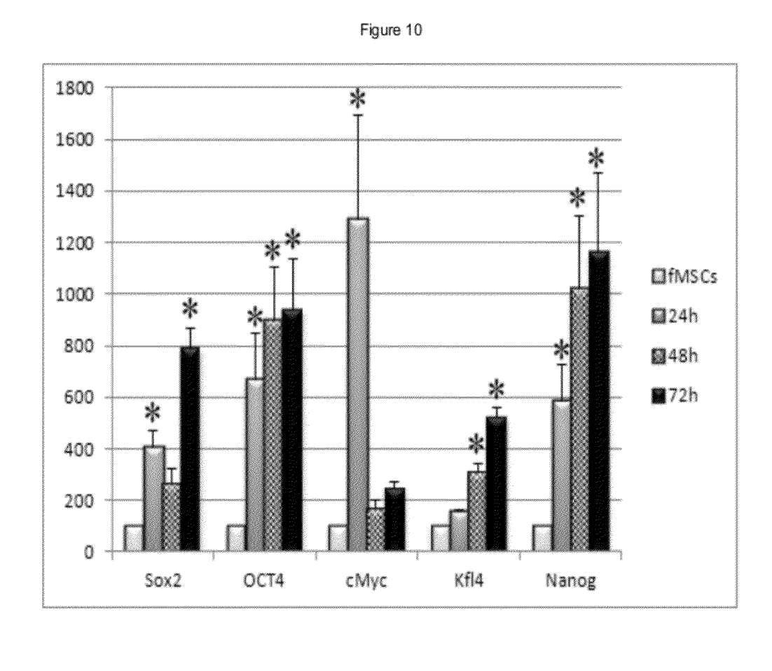

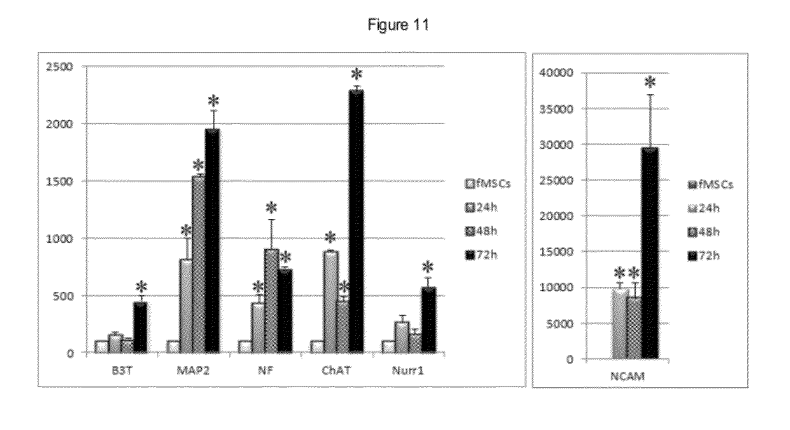

ELISA studies demonstrated that NI-fMSCs released neurotrophic factors GDNF (144.4±7.51 pg/ml//1×10⁵/day) and NGF (377.1±90.11 pg/ml//1×10⁵/day). Gene expression studies by real time RT-PCR showed that native fMSCs expressed several pluripotent markers such as Oct-4, Nanog, Sox2, cMyc, and KLF4. With neural induction, expression levels of Sox2, Klf4, Nanog, and Oct4 were gradually increased during the next three days in culture (FIG. 10). Expression level of cMyc was highest at 24 h and then decreased during the next 2 days of the treatment but still stayed high in comparison to control (FIG. 10). Expression levels of most of the immature and mature neural markers also were gradually increased and reached to their highest level at 72 h of the treatment (FIG. 11).

The expression of several genes associated with pluripotence and most of the neural genes in unmodified and NI-fMSCs was confirmed by Western blot. The expression levels of pluripotent markers Sox-2, cMyc, and Nanog (except of Oct4 and Klf4) in NI-hMSCs gradually increased and became significantly different from unmodified hMSCs after 48 h and 72 h of the treatment (FIG. 7). The lack of the expression of Oct-4 and Klf4 indicates that either the expression level of these proteins was very low or they were expressed only at the mRNA level. Western blot studies also revealed a gradual increase of neuroectoderamal gene expression in NI-fMSCs, which became significantly different from the control after 48-72 h of treatment (FIG. 8). This increase in neural gene expression was accompanied with significant morphological changes of fMSCs into neural phenotypes. Interestingly, neural transformation rate of fMSCs was much faster (only 3 days) in contrast to mouse and human MSCs, which usually takes about two weeks.

### 4. Conclusions

Feline MSCs manipulated with a neural induction protocol in accordance with the invention turned into neural-like cells more easily and efficiently compared to mice and human MSCs. The higher plasticity of fMSCs is likely explained by the moderate expression of several pluripotent and neural genes in unmodified fMSCs.

All patents, patent applications, and publications mentioned in the specification are indicative of the levels of those of ordinary skill in the art to which the invention pertains. Each patent, patent application, and publication cited herein is hereby incorporated by reference in its entirety for all purposes regardless of whether it is specifically indicated to be incorporated by reference in the particular citation.

All of the compounds, compositions, and methods disclosed and claimed herein can be made and executed without undue experimentation in light of the present disclosure. Moreover, it is intended to obtain rights which include alternative and/or equivalent embodiments to the extent permitted, including alternate, interchangeable, and/or equivalent structures, functions, ranges, or steps to those claimed, whether or not such alternate, interchangeable and/or equivalent structures, functions, ranges, or steps are disclosed herein, and without intending to publicly dedicate any patentable subject matter, as it is intended that all patentable subject matter disclosed herein eventually be the subject of patent claims.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Also, the invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Furthermore, while the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

I claim:

1. A method for reprogramming mammalian mesenchymal stem cells, comprising culturing a cellular composition comprising mammalian mesenchymal stem cells under culturing conditions that include an effective amount a first epigenetic modifying agent, a second epigenetic modifying agent, and a cAMP elevating agent for a period sufficient to allow reprogramming of at least a portion of the mammalian mesenchymal stem cells, wherein the mammalian mesenchymal stem cells optionally are human mesenchymal stem cells, optionally human mesenchymal stem cells derived from bone marrow, peripheral blood, muscle, vasculature, skin, adipose tissue, or umbilical cord.

2. A method according to claim 1, wherein the first epigenetic modifying agent is an inhibitor of DNA methylation, optionally either 5-aza-2′-deoxycytidine (5azadc) and RG-108.

3. A method according to claim 2, wherein the second epigenetic modifying agent is an inhibitor of histone deacetylation, optionally Trichostatin A.

4. A method according to claim 1, wherein the cAMP elevating agent is selected from a hydrolysis-resistant form of cAMP, optionally BrcAMP; an adenylate cyclase activator, optionally Forskolin; and an inhibitor of cAMP phosphodiesterase, optionally either IBMX or Rolipram.

5. A method according to claim 1, wherein a first epigenetic modifying agent is RG-108, optionally present in a concentration ranging from about 1 μM to about 10 μM, the second epigenetic modifying agent is Trichostatin A, optionally present in a concentration ranging from about 50 nM to about 500 nM, and the cAMP elevating agent is selected from the group consisting of 8-BrcAMP (optionally present in a concentration ranging from about 100 μM to about 500 μM) and Rolipram (optionally present in a concentration ranging from about 0.1 μM to about 10 μM).

6. A method according to claim 1, wherein the cells are cultured for about 5 to about 20 days.

7. A method according to claim 1, wherein the culturing conditions further comprise an amount of bFGF effective for neural induction.

8. An isolated population of reprogrammed mammalian mesenchymal stem cells produced according to the method of claim 1.

9. A method of generating a population of neural stem cells from mammalian mesenchymal stem cells, comprising culturing a cellular composition comprising mammalian mesenchymal stem cells under culturing conditions that include an effective amount a first epigenetic modifying agent, a second epigenetic modifying agent, a cAMP elevating agent, and bFGF for a period sufficient to generate a cell population at least of portion of which comprises neural stem cells, wherein the mammalian mesenchymal stem cells optionally are human mesenchymal stem cells, optionally human mesenchymal stem cells derived from bone marrow, peripheral blood, muscle, vasculature, skin, adipose tissue, or umbilical cord.

10. A method according to claim 9, wherein the first epigenetic modifying agent is an inhibitor of DNA methylation, optionally either 5-aza-2′-deoxycytidine (5azadc) and RG-108, the second epigenetic modifying agent is an inhibitor of histone deacetylation, optionally Trichostatin A, and the cAMP elevating agent is selected from a hydrolysis-resistant form of cAMP, optionally BrcAMP; an adenylate cyclase activator, optionally Forskolin; and an inhibitor of cAMP phosphodiesterase, optionally either IBMX or Rolipram.

11. A method according to claim 9, wherein the first epigenetic modifying agent is RG-108, optionally present in a concentration ranging from about 1 μM to about 10 μM.

12. A method according to claim 9, wherein the second epigenetic modifying agent is Trichostatin A, optionally present in a concentration ranging from about 50 nM to about 500 nM.

13. A method according to claim 9, wherein the concentration of bFGF ranges from about 5 ng/mL to about 50 ng/mL.

14. A method according to claim 9, wherein the cAMP elevating agent comprises 8-BrcAMP, optionally from about 100 μM to about 500 μM BrcAMP, or Rolipram, optionally from about 0.1 μM to about 10 μM Rolipram.

15. A method according to claim 1, wherein the cells are cultured for about 5 to about 20 days.

16. An isolated cell population comprising neural stem cells produced according to the of method claim 9, wherein optionally at least about 50%, about 80%, or about 90% or more of the cells in the cell population are neural stem cells.

17. A method of treating a spinal cord injury or a central nervous system disease or condition in a mammalian subject, comprising administering to a subject having or suspected of having a spinal cord injury or central nervous system disease or condition an isolated cell population according to claim 16.

18. A method according to claim 17, wherein the cell population is administered intravenously, intrathecally, or directly into the injured spinal cord or central nervous system tissue.
